(19) United States
(12) Patent Application Publication
(10) Pub. No.: US 2017/0362184 A1
(43) Pub. Date: Dec. 21, 2017

(54) PRO-SURVIVAL COMPOUNDS

(71) Applicants: University Court of The University of Glasgow, Glasgow (GB); Heriot-Watt University, Edinburgh (GB)

(72) Inventors: Joanne Mountford, Glasgow (GB); Graeme Milligan, Glasgow (GB); Dave Adams, Edinburgh (GB)

(21) Appl. No.: 15/534,537

(22) PCT Filed: Dec. 11, 2015
(86) PCT No.: PCT/GB2015/053884

§ 371 Date: Jun. 9, 2017

(30) Foreign Application Priority Data
Dec. 11, 2014 (GB) 1422060.2

(51) Int. Cl. C07D 235/30; C12N 5/071; C07D 403/12; C07D 401/12; C07D 405/12; C07D 413/12; C07D 417/12

(52) U.S. Cl. CPC C07D 235/30; C07D 413/12; C12N 5/0602; C07D 417/12; C07D 401/12; C07D 405/12; C07D 403/12; C12N 2501/999

(57) ABSTRACT

Disclosed herein are a class of compounds useful in cell culture, in particular, the in vitro culture of stem cells. The compounds have been found to promote the survival and/or maintenance of stem cells in (or during) culture and/or throughout passage.

### FIELD

The present invention relates to the use of certain compounds in the growth and/or culture of stem cells, such as pluripotent stem cells. The invention also relates to methods for culturing stem cells and compositions and media for cell culture comprising the compounds described herein.

### BACKGROUND

Stem cells such as human pluripotent stem cells (hPSC) e.g. human embryonic stem cells (hESC) and induced pluripotent stem cells (iPSC) are valuable tools for investigations within a number of scientific fields including developmental biology, toxicology, pharmacology and regenerative medicine. Stem cells have an extensive (sometimes unlimited) capacity for self-renewal which allows the expansion and differentiation of clinically relevant cell numbers from a relatively small supply of starting material. Pluripotent stem cells (including hPSC) retain the capacity to differentiate into all the somatic cell types within the human body.

However a major obstacle to this area of research is the development of consistent and scalable culture systems that allow sufficient expansion of stem cells without loss of the stem cell identity (i.e. the multi- or pluripotent phenotype). For example, hPSC are particularly susceptible to loss of viability and/or cell death upon enzymatic disaggregation wherein approximately 80% of the hPSC may lose viability and/or begin to die.

Recent efforts to promote the (prolonged) survival of hESC in culture systems have focussed on the Rho associated coiled-coil kinase (ROCK) inhibitor Y27632 (Watanabe et al, Nat. Biotechnol. 2007, 25(6), 681-686). A recent study has also identified 18 small molecule ROCK inhibitors that support the enzymatic passage of hPSC (Andrews et al, 2010).

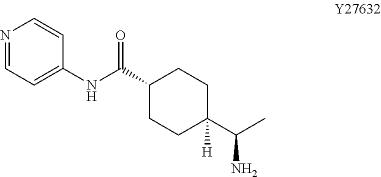 [SMILES: C[C@@H](N)[C@H]1CC[C@H](C(=O)Nc2ccncc2)CC1]

However, there is increasing evidence that the use of ROCK inhibitors such as Y27632, can lead to an increased risk of karyotypic instability, a decrease in proliferative capacity and a reduced capacity to differentiate in to specific cell types such as haematopoietic cell types (Zweigerdt et al, 2011; Singh et al, 2010; Liu et al, 2012; Yung et al, 2011).

Despite the disadvantages of using ROCK inhibitors in stem cell culture systems, ROCK inhibitors such as Y27632 remain widely used in the field. Therefore there is a need for alternative compounds with an ability to promote long term survival in culture without significant loss of the pluripotent phenotype. Compounds of this type may find particular application in stem cell culture.

A number of studies have been performed with the aim of discovering alternative pro-survival compounds, however, despite utilising high-throughput approaches, a viable alternative to Y27632 has yet to be identified (Wong et al, 2007; Yamaguchi and Wang, 2001; Xu et al, 2010).

### SUMMARY

The present invention resides in the identification of a class of compounds useful in cell culture, in particular, the in vitro culture of stem cells. The compounds provided by this invention have been found to promote the survival and/or maintenance of stem cells in (or during) culture and/or throughout passage.

The compounds provided by this invention may be referred to as (stem) cell “pro-survival compounds” which, as stated (and explained in more detail below), find particular application in the culture of stem cells.

In contrast to those pro-survival compounds known in the art and without wishing to be bound by theory, the compounds described herein appear to have a different mode of action which does not rely on the inhibition of ROCK; thus they are ROCK independent pro-survival compounds. Methods and uses which exploit the compounds of this invention may mitigate, reduce and/or eliminate one or more of the disadvantages associated with the use of ROCK inhibitors during stem cell culture, such as increased risk of genetic instability, decreased proliferative capacity and reduced capacity to differentiate into specific cell types.

In particular, the compounds described herein may be useful in the culture of stem cells which may be differentiated into haematopoietic cell types.

As used herein, the term “pro-survival” relates to one or more agent(s), factor(s) or compound(s) which maintain, facilitate, enhance and/or promote the growth of stem cells in vitro. The pro-survival compounds of this invention may, in use, allow or facilitate, the prolonged survival of stem cells in culture and over (or through) a number of passages. Stem cells which survive in vitro culture may remain viable and/or retain a multipotent phenotype over all or part of the period of culture and/or a number (for example one, two or more) passages.

Stem cells may be referred to as progenitor cells and may be further described as either pluripotent, multipotent or totipotent. For the avoidance of doubt, the term “stem cells” embraces all types of stem and progenitor cell described herein and the term “multipotent” encompasses both pluripotent and totipotent cells. Moreover, throughout this specification the term “comprising” is used to denote that embodiments of the invention “comprise” the noted features and as such, may also include other features. However, in the context of this invention, the term “comprising” may also encompass embodiments in which the invention “consists essentially of” the relevant features or “consists of” the relevant features.

The invention provides, or relates to compounds according to formula (I):

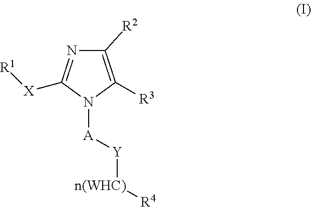

wherein:

R¹ is H, aryl, substituted aryl, alkyl, CH(CH₃)R⁵, or CH₂R⁵;

R⁵ is aryl, substituted aryl, heteroaryl, substituted heteroaryl or heterocyclic;

X is NZ, O, CH₂ or S, or alternatively X may not be present;

Z is H or alkyl;

R² and R³ are each independently selected from H, alkyl, aryl, substituted aryl, heteroaryl, substituted heteroaryl, or alternatively R² and R³ together form an aromatic or heteroaromatic ring, optionally comprising one or more substituents;

A is CH₂, CH₂CH₂ or CH(CH₃);

Y is C(═O)NZ′, C(═O)O, SO₂NH, NHC(═O), C(═O)NHC(═O), NH, or O;

Z′ is H, alkyl, alkoxyalkyl, or wherein Z′ and R⁴ together form a 5- or 6-membered ring heterocyclic group with at least one N atom;

n is 0, 1, 2 or 3;

W is H or alkyl, wherein when n is greater than 1, each W is independently selected from H or alkyl;

R⁴ is H, alkyl, cycloalkyl, aryl, substituted aryl, heteroaryl, substituted heteroaryl, OR⁶, heterocyclic, alkylamino, C≡N or C(═O)R⁷;

R⁶ is H, alkyl, haloalkyl, aryl, alkoxyalkyl; and

R⁷ is hydroxy, alkoxy, amino or alkylamino.

As used herein, “alkyl” refers to straight or branched chain saturated hydrocarbons. For example, hydrocarbon chains containing from 1 to 6 carbon atoms, or 1 to 5 carbon atoms, or preferably 1 to 4 carbon atoms. As used herein, C₁-C₄ alkyl refers to a straight or branched chain hydrocarbon containing from 1 to 4 carbon atoms. Representative examples of alkyl groups include, but are not limited to, methyl, ethyl, n-propyl, iso-propyl, n-butyl, sec-butyl, iso-butyl, tert-butyl etc.

The term “haloalkyl” refers to an alkyl group as defined herein, in which one or more hydrogen atoms have been replaced with a halogen atom, such as a fluoro or chloro group. For example, a haloalkyl group may be a perfluoroalkyl group (e.g. CF₃).

“Aryl” as used herein may be a single or fused ring system having one or more aromatic rings. Representative examples include phenyl (—C₆H₅) and naphthyl.

“Substituted aryl” as used herein refers to an aryl group as defined herein which comprises one or more substituents. The substituents may comprise, but are not limited to, C₁-C₄ alkyl (e.g. methyl or ethyl groups), C₁-C₄ alkoxy groups (e.g. methoxy or ethoxy groups), halo groups, cyano groups, alkylamino groups, nitro groups, or substituted alkyl groups (e.g. halo-substituted alkyl, such as perfluorinated alkyl).

“Heteroaryl” as used herein refers to a single or fused ring system having one or more aromatic rings containing 1 or more 0, N and/or S heteroatoms. Representative examples of heteroaryl groups may include, but are not limited to, pyrrole, furan, thiophene, pyrazole, imidazole, oxazole, isoxazole, oxadiazole, thiazole, pyridine, pyrimidine, pyridazine, pyrazine, indole, benzofuran, benzothiazole, benzimidazole, indazole, benzoxazole, benzisoxazole etc.

“Substituted heteroaryl” as used herein refers to a heteroaryl group as defined herein which comprises one or more substituents. The substituents may comprise, but are not limited to, C₁-C₄ alkyl, C₁-C₄ alkoxy groups, halo groups, cyano groups, alkylamino groups, nitro groups, or substituted alkyl groups (e.g. halo-substituted alkyl, such as perfluorinated alkyl).

“Heterocyclic” groups may comprise at least 1 heteroatom. Heterocyclic groups may be aliphatic heterocyclic groups. Heteroatoms may be 0 and/or N. Heterocyclic groups may be 5- or 6-membered ring heterocycles containing 1 or more heteroatoms. Representative examples include, but are not limited to, piperidine, piperazine, N-alkylpiperazine, morpholine, dioxane, oxane, tetrahydrofuran, or tetrahydropyran.

“Alkoxy” as used herein refers to an alkyl group, as defined above, appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy etc.

“Alkoxyalkyl” as used herein refers to an alkyl group comprising an alkoxy group appended to the alkyl group through an oxy group, —O—. Representative examples of alkoxyalkyl include, but are not limited to, methoxyethyl, ethoxyethyl, t-butoxyethyl etc.

“Alkylamino” as used herein refers to an alkyl group, as defined above, appended to the parent molecular moiety through an amino group (—N—). The amino group may comprise one or more alkyl groups and may be a monoalkylamino or a dialkylamino group. Representative examples of alkylamino include, but are not limited to dimethylamino and monomethylamino.

In some embodiments, X may be a hydrogen bond donor group, e.g. X may be NH. In certain embodiments, X may be NZ, wherein Z may be a C₁-C₄ alkyl group, such as a methyl group.

In some embodiments, R¹ may be H or a C₁-C₄ alkyl group. In some embodiments, R¹ may be CH₂R⁵ or CH(CH₃)R⁵.

R⁵ may be an aryl group, such as a phenyl group, or a substituted aryl group, such as a substituted phenyl group. Representative examples of substituent groups are described herein and may include, for example, a halo-substituted phenyl group.

Alternatively R⁵ may be a heteroaryl or heterocyclic group containing at least one nitrogen or oxygen atom. For example, R⁵ may be a 6-membered ring heteroaryl group, such as pyridyl. In other embodiments, R⁵ may be a 6-membered ring aliphatic heterocyclic group, such as tetrahydropyran.

In some embodiments, A may be CH₂.

In some embodiments, Y may comprise a functional group with hydrogen bond donor and/or acceptor ability. For example, Y may be an amido group (C(═O)NZ′). In these embodiments, Z′ may be H.

Moiety n may be 2. In this and other cases, at least one W may be an alkyl group. For example, at least one W may be a C₁-C₄ alkyl group, e.g. a methyl group.

R⁴ may be a heteroaryl group, or a substituted heteroaryl group, optionally a C₁-C₄ alkyl substituted heteroaryl group. R⁴ may comprise a 5- or 6-membered heteroaryl ring. For example, R⁴ may be an isoxazole or a C₁-C₄ alkyl substituted isoxazole, or R⁴ may be pyrazine. In particular, R⁴ may be a methyl-substituted isoxazole.

Alternatively, R⁴ may be an alkoxy group, such as a C₁-C₄-alkoxy group. For example, R⁴ may be a methoxy or ethoxy group.

In some cases, R² and R³ together may form a 6-membered aromatic ring or heteroaromatic ring to provide a fused ring core. The 6-membered aromatic or heteroaromatic ring may comprise one or more substituents as described herein.

In those cases where R² and R³ together form a 6-membered aromatic ring, the compound may comprise a benzimidazole core as shown in formula (II):

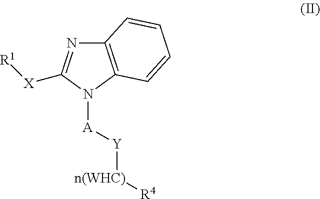

wherein R¹, X, A, Y, W, n and R⁴ are as defined for formula (I).

Further, whilst not being bound by theory, the inventors believe that a hydrogen bond acceptor site (HAcc) at the site X′ shown in formula (III) may assist in target binding of the compound and so enhance the pro-survival effect. Compounds of formula (III) have been found to be surprisingly effective at promoting the preservation of a multi- or pluripotent phenotype in stem cells even at low concentrations:

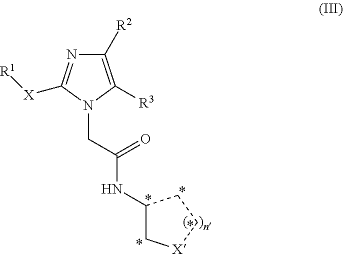

wherein R¹, R², R³ and X are as defined for formula (I);

X′ is a hydrogen bond acceptor site;

n′ is 1 or 2;

the dotted lines indicate that the hydrogen bond acceptor site may be incorporated into a heteroaryl or heterocyclic ring, or alternatively the dotted lines may not be present and the hydrogen bond acceptor site may be present on an acyclic side chain, in either case the cyclic or acyclic side chain may comprise one or more alkyl substituents; and

wherein one or more of the starred carbon atoms (*) may be substituted with a heteroatom.

The hydrogen bond acceptor site may comprise a heteroatom (such as a nitrogen or oxygen atom). The heteroatom may be comprised in a heteroaryl group (e.g. pyrazine group or isoxazole), or in an acyclic side chain (e.g. a C₁-C₄ alkoxy group).

By way of further example are shown compounds of formula (IV):

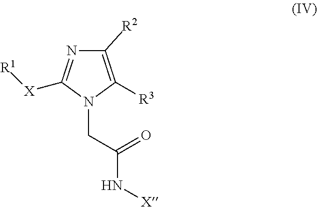

wherein R¹, R², R³ and X and are as defined for formula (I); and

X″ is a heteroaryl, substituted heteroaryl or an alkoxyalkyl group.

X″ may be a 5- or 6-membered heteroaryl ring. X″ may be a heteroaryl group comprising at least one nitrogen atom. Representative examples of such heteroaryl groups may include, but are not limited to, an isoxazole or a pyrazine group. X″ may be a heteroaryl group comprising one or more substituents. Substituents may comprise C₁-C₄ alkyl groups, such as a methyl group.

In embodiments where X″ is an alkoxyalkyl group, alkoxyalkyl is a C₁-C₄ alkoxyl-C₁-C₄ alkyl group. For example, an alkoxyethyl or alkoxy methylethyl group. Representative examples of C₁-C₄ alkoxy groups include, but are not limited to, a methoxy or ethoxy group.

Compounds of this invention may include those in which X″ is selected from:

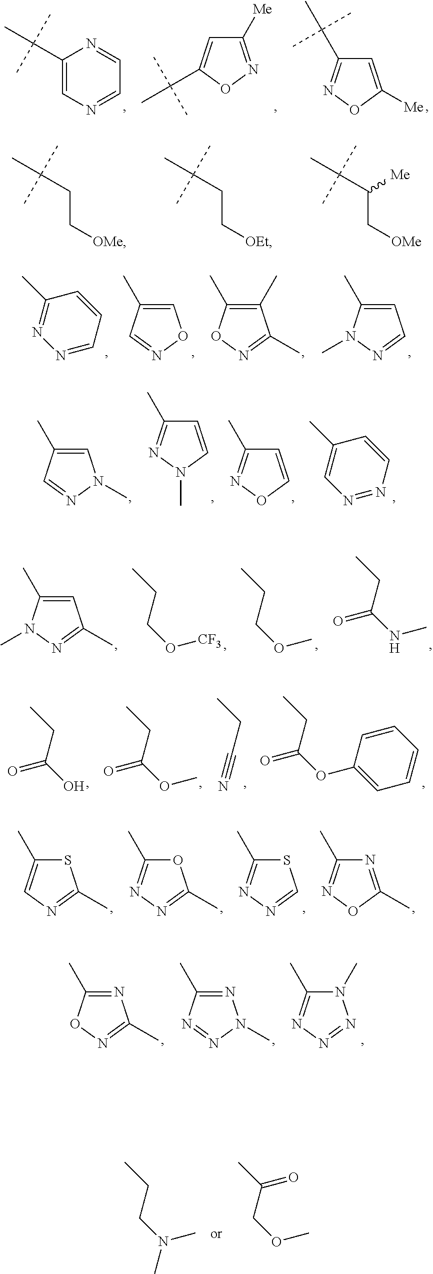

It will be appreciated that the compounds described herein may exist in different stereoisomeric forms. For example, the stereochemistry at the methyl substituted position in the acyclic side chain above is indicated by a wavy bond. The wavy bond indicates that the connected stereogenic centre may be either (R)- or (S)-configured, according to the terms of standard chemical nomenclature used in the art. The present invention includes within its scope the use of both stereoisomeric forms, or the use of a mixture of stereoisomers, including the individual enantiomers of the compounds as well as wholly or partially racemic mixtures of such enantiomers. For example, X″ may be:

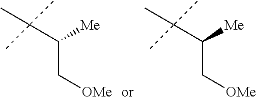

As discussed previously, R² and R³ together may form an aromatic or heteroaromatic ring, optionally comprising one or more substituents. For example, R² and R³ together may form a 6-membered aromatic ring and compounds of formula (IV) may comprise a benzimidazole core.

In view of the above, a first aspect of the invention provides compounds according to formula (I), (II), (III) or (IV).

The first aspect of the invention may relate to novel compounds according to formula (I), (II), (III) or (IV).

The first aspect of this invention may not relate to one or more of the compounds shown in Appendix 1.

In particular, the invention relates to those compounds which:

promote the survival of stem cells in in vitro culture
(ii) promote the survival and/or facilitate the maintenance of the multipotent phenotype of stem cells in culture; and/or
(ii) promote the survival and/or facilitate the maintenance of the pluripotent/multipotent phenotype of enzymatically or otherwise dissociated stem cells in culture and/or throughout subsequent passage.

Useful compounds of this invention (that is “active” compounds or compounds which satisfy one or more of the features listed as (i), (ii) and (iii) immediately above) may comprise carefully positioned hydrogen bonding features. For example, useful compounds of this invention may be characterised by:

X″ (with reference to formula IV above) is a 5- or 6-membered heterocyclic ring and with a hydrogen bond acceptor feature (for example, N or 0) in a 1,3-relationship with the point of connection to the amide nitrogen; or

X″ (again with reference to formula IV above) is a small aliphatic chain with a hydrogen bond feature (e.g. 0) connected to the amide nitrogen through two chain atoms and terminating in a small group (e.g. as X″═—CH2-CH2-O-Me, —CH2-CH2-O-Et).

For example, the invention may relate to one or more of the compounds presented in Table 1 (below), all of which exhibit “pro-survival” activity.

TABLE 1

| | Internal Designation |
|---|---|
| Name | Number¹ |
| 2-(2-(benzylamino)-1H-benzo[d]imidazol-1-yl)-N- | SC332 |
| (5-methylisoxazol-3-yl)acetamide | |
| 2-(2-(benzylamino)-1H-benzo[d]imidazol-1-yl)-N- | SC330 |
| (3-methylisoxazol-5-yl)acetamide | |
| 2-(2-(benzylamino)-1H-benzo[d]imidazol-1-yl)-N- | SC050 |
| (2-methoxyethyl)acetamide | |
| 2-(2-(benzylamino)-1H-benzo[d]imidazol-1-yl)-N- | SC119 |
| (pyrazin-2-yl)acetamide | |
| 2-(2-(benzylamino)-1H-benzo[d]imidazol-1-yl)-N- | SC839 |
| (2-ethoxyethyl)acetamide | |
| (R)-2-(2-(benzylamino)-1H-benzo[d]imidazol-1-yl)- | SC321 |
| N-(1-methoxypropan-2-yl)acetamide | |
| 2-(2-(benzylamino)-1H-benzo[d]imidazol-1-yl)-N- | SC408 |
| (5-methyl-1,3,4-oxadiazol-2-yl)acetamide | |
| 2-(2-(benzylamino)-1H-benzo[d]imidazol-1-yl)-N- | SC554 |
| (5-methyl-1,2,4-oxadiazol-3-yl)acetamide | |
| 2-(2-(benzylamino)-1H-benzo[d]imidazol-1-yl)-N- | SC585 |
| (3-methyl-1,2,4-oxadiazol-5-yl)acetamide | |

¹As used in the examples section

The compounds of this invention may promote the survival of stem cells for prolonged periods and/or over a number of passages in culture. The compounds may further be used to ensure that the multipotent phenotype of stem cells is maintained during culture and/or over a number of passages. As explained elsewhere, the terms “survival” or “survive” embrace the maintenance of cell viability and/or multipotency during periods of culture and/or passage. Thus, stem cells which “survive” in culture may remain multipotent and/or viable over a number of passages and/or for longer than expected. Stem cells subjected or exposed to the various compounds, compositions, methods and/or uses of this invention may survive for longer (i.e. be passaged more extensively and/or be able to withstand (remaining viable and/or multipotent during) prolonged culture), as compared to stem cells not subjected to or contacted with the various compounds, compositions, methods and uses of this invention.

For example, stem cells which are subject to the compounds, compositions, methods and/or uses of this invention may be passaged, without loss or substantial loss of any multipotent phenotype and/or viability, at least 1, 2, 5, 10, 15, 20, 25, 30, 35, 40, 45, 50 or more times. Stem cells contacted with a compound or composition of this invention or subjected to a method or use described herein may retain a particular phenotype, for example a multipotent phenotype, throughout all or part of the period of culture and/or passage.

The term “stem cells” as used herein may include any cell which is able to self-renew and indefinitely divide—cells of this type may be described as “immortal”. In addition, when cultured under suitable conditions and/or contacted with, or exposed to, particular compounds and/or conditions, stem cells may differentiate into one or more of the specialised cell types which form embryonic and/or adult tissues.

Stem cells may be totipotent in nature and one of skill will appreciate that totipotent cells may be capable of generating a complete viable organism as well as any given specialised cell type. Stem cells may be pluripotent—cells of this type are not capable of generating a complete viable organism, but are able to differentiate to one or more (sometimes any) specialised cell type. As such, the present invention may be applied to any type of totipotent and/or pluripotent stem cell. Collectively, such cells shall be referred to hereinafter as “multipotent” stem cells.

The term “stem cells” may encompass animal, mammalian (including primate/human), embryonic, foetal, adult and/or induced pluripotent (iPS) stem cells.

The term “stem cells” may further encompass progenitor cells of any type. For example, the stem cells mentioned herein may be mammalian cells; for example, the term “stem cells” may be applied to human and/or non-human stem cells of all types. By way of example the compounds, compositions and/or uses/methods of this invention may be applied to stem cells from stem cells derived or obtained from, or provided by, primates, ungulates, ruminants and/or rodents (specifically, sheep, pigs, cattle, goats, horses, rats and mice).

Stem cells to which the various aspects and embodiments of this invention may be applied, may be characterised by the presence of one or more markers selected from the group consisting of but not limited to: ABCG2; ACE; ALCAM; Alkaline Phosphatase; beta-III Tubulin; BMP-2; BMPR-IA/ALK-3; BMPR-IB/ALK-6; BMPR-II; E-Cadherin; CCR4; CD9; CD71; CD90; CD90/Thy1; Cripto; CXCR4; DPPAS/ESG1; Endoglin/CD105; FABP1; FABP2; FGF-4; FGF R4; FoxD3; FoxP3; Frizzled-9; GAD1/GAD67; GATA-4; GATA-6; GDF-3; Glut1; HNF-3 beta; Integrin alpha 6/CD49f; Integrin beta 1/CD29; Lefty; MAP2; Musashi-1; Nanog; NCAM-L1; Nectin-2/CD112; Nestin; NeuroD1; Nodal; Noggin; NF-L; NF-M; Nucleostemin; Otx2; Oct3/4; PAX6; Podocalyxin; Prominin 2; ROBO3; Sca-1; SCF R/c-kit; SHH; SOX2; SOX7; SOX17; SPARC; SSEA-1; SSEA-3; SSEA-4; STAT3; STRO-1; TP63/TP73L; TRA-1-81; TRA-1-60; Tyrosine Hydroxylase; gamma-Secretase; alpha-Secretase; beta-Secretase; beta-III tubulin; alpha-Fetoprotein; beta-Catenin; Vimentin and VCAM-1. Collectively, these markers may each be referred to as stem cell markers, and references in this specification to one or more “stem cell markers” may therefore encompass one or more of the abovementioned markers. One of skill will appreciate that to identify or detect a stem cell, a cell may be probed (using, for example antibodies or other agents capable of binding one or more of the listed stem cell markers) for the presence of one or more of the stem cell markers mentioned herein.

It should be understood that compounds, compositions, methods and uses of this invention may be applied to cells differentiated or derived from any of the stem cells described above. A more detailed description of some specific types of stem cell is provided below.

The term “stem cells” may embrace embryonic stem cells (ESC), for example, mammalian and/or human embryonic stem cells (hESC). ESCs may be derived from early stage embryos and in particular from the inner cell mass of the developing morula or blastocyst. Embryonic stem cells, for example those derived from embryos in the stages immediately following conception (and for a short time thereafter), may be totipotent (capable of generating a complete viable organism as well as any given specialised cell type). Embryonic stem cells derived from later stage embryos (i.e. from the inner cell mass of a developing blastocyst) may be pluripotent (not capable of generating a complete viable organism, but capable of differentiating to any specialised cell type).

The term “stem cells” may further encompass hESCs and other cell lines obtained from an embryo without destruction of the embryo, as described, for example, in Chung et al (Cell Stem Cell, vol 2, issue 2, 113-117, 2008). Stem cells disclosed in this specification may also be generated using the methods described by Chung et al., (2006) which methods involve taking a blastomere cell from an early stage embryo prior to formation of the blastocyst (at approximately the 8-cell stage) and co-culturing this cell with established stem cell lines to generate a fully competent stem cell line. Thus the various compounds, compositions, methods and uses described herein may be applied to stem cells obtained by the methods described by Chung et al (2006, 2008) and Tachibana et al (2013, Cell 153 (6), p 1228-1238 and Cell 154 (2), p 465-466).

Markers of embryonic stem cells to which this invention may be applied may include, for example, ABCG2, Alkaline Phosphatase, E-Cadherin, CCR4, CD9, Cripto, DPPA5/ESG1, FGF-4, FGF R4, FoxD3, FoxP3, GDF-3, Integrin alpha 6/CD49f, Integrin beta 1/CD29, Lefty, Nanog, Oct3/Oct4, Podocalyxin, SOX2, SPARC, SSEA-1, SSEA-3, SSEA-4; STAT3; TRA-1-81 and TRA-1-60.

The term “stem cells” may also be taken to refer to stem cells, for example pluripotent cells, derived from any of the three primary germ layers (ectoderm, mesoderm and endoderm) which develop during the process of gastrulation. Cells derived from these layers may express one or more markers which may be used as a means of identification. By way of example, ectoderm germ layer may express markers, including, for example, Otx2, Nestin, TP63/TP73L, beta-III Tubulin, SHH, and PAX6. Ectoderm has the potential to form cell types such as neurons and early neuronal lineage markers include ACE, ALCAM, CD90/Thy1, GAD1/GAD67, Glut1, MAP2, NCAM-L1, Nectin-2/CD112, NeuroD1, NF-L, NF-M, ROBO3, gamma-Secretase, alpha-Secretase, beta-Secretase, beta-III tubulin, Tyrosine Hydroxylase. Neural stem cell markers include ABCG2, CXCR4, FGF R4, Frizzled-9, Musashi-1, Nestin, Noggin, Nucleostemin, Prominin 2, SOX2, Vimentin. Mesoderm has the potential to form haematoendothelial, cardiovascular and/or mesenchymal cells including, for example, blood cells, endothelium, cardiomyocytes, smooth muscle and fibroblasts. Early mesodermal lineage markers may include, for example, one or more of CD56 (mesoderm cell populations may be CD336^{-ve} and CD56⁺), GSC, MIXL1, CXCR4, FOXF1, VENTX and KDR. Markers of early endodermal cells include, for example, FABP1, FABP2, GATA-4, HNF-3 beta (collectively referred to as definitive endodermal stem cells markers) as well as those markers for primitive endoderm such as alpha-Fetoprotein, beta-Catenin, GATA-4, SOX17 and SOX7.

The invention may also be applied to “adult” stem cells—cells of this type may be taken to be stem cells obtained from adult animals and or adult (or developed/differentiated) tissue (including adult humans and/or human (adult) tissue). However, it should be understood that the term “adult” also includes stem cells derived from neonatal, infant, juvenile and/or adolescent animals. Adult stem cells may be sourced from any suitable tissue, including bone marrow and/or specialised structures such as, for example hair follicles, skin, teeth and the like.

Stem cells to which this invention may be applied may be obtained from a variety of sources including, for example, embryonic animals (including human embryos), said embryos being either aborted or created as part of a fertility program. Alternatively, it may be possible to obtain stem cells from established stem cell lines, thus avoiding the use of mammalian, particularly human, embryos. By way of example, stem cells may be obtained from the H1, H9 and/or RC9, RC111 cell lines.

Alternatively, the methods of Meissner & Jaenisch (2006) may be used to obtain stem cells to which this invention may be applied. In these methods, the cdx2 gene is silenced in the donor nucleus during the process of nuclear transfer to prepare a reconstructed embryo from which a line of embryonic stem cells is derived. The cdx2 gene is turned back on in the isolated blastocyst cell taken from the embryo which is used to prepare the cell line. This is an example of, so-called, “alternative nuclear transfer” where the embryo is not capable of implantation but the stem cell line derived therefrom is fully competent.

The term “stem cells” may also encompass cells otherwise known as induced pluripotent stem cells (iPS). These are re-programmed adult somatic cells which have been modified to express certain factors (such as transcription regulators) and, as a consequence, become pluripotent and thus capable of differentiating to any other specialised cell type.

One of skill will appreciate that in certain cell culture protocols, it is necessary to dissociate clumped or aggregated cells—for example stem cell embryoid bodies. The dissociation may be mechanical and/or chemical based and thus the term “stem cell” may therefore be applied to any mechanically or chemically dissociated stem cells. For example, the term “stem cell” may embrace enzymatically dissociated stem cells.

For the avoidance of doubt, the various compounds, compositions, methods and uses of this invention may be applied to any of the stem cell types described herein. Moreover, it should be understood that the term “stem cell” may encompass a population of stem cells. Thus the methods, uses, compounds and/or compositions of this invention may be applied to populations of stem cells. Within a population of stem cells subjected to a method, use, compound or composition of this invention, one or more of the cells may be maintained or survive culture and/or passage. One of skill will appreciate that on occasion, not all of the cells will survive culture and/or passage—some cells may naturally apoptose and/or loose viability and others may become less plastic. Nevertheless, a population of cells subjected to the methods, compounds or compositions of this invention may contain cells which survive longer than cells of a population which have not been subjected to the methods compounds and/or compositions of this invention.

A second aspect of this invention provides a composition, comprising one or more compounds according to formula (I), (II), (III) or (IV). The composition may be a liquid (aqueous) or solid (for example lyophilized) composition and may comprise one or more diluents, excipients and/or carriers. For example, a composition of this invention may take the form of a supplement comprising one or more of the pro-survival compounds described herein, wherein the supplement is, for example, formulated for addition to a cell culture medium—in particular, a stem cell culture medium. The composition may be a sterile or pharmaceutical grade composition, in which case any diluents, excipients and/or carriers may be sterile and/or pharmaceutically acceptable.

The composition may take the form of (or comprise) a cell culture medium and in a third aspect the invention provides a cell culture medium comprising one or more compounds according to formula (I), (II), (III) or (IV). A cell culture medium of this invention may be a stem cell culture medium.

Cell culture media according to the third aspect of this invention may comprise a home-made or commercially available base medium suitable for the culture, maintenance and/or expansion of cells and/or stem cells. Media of this type may be supplemented with one or more of the compounds of this invention and may further comprise, for example, compounds and/or molecules which facilitate the maintenance and/or expansion of stem cells. The base medium may comprise an Iscove's Modified Dulbecco's Media (IMDM) or Dulbecco's Modified Eagle Medium (DMEM)/F12 based medium. By way of example, the base medium may comprise one or more selected from the group consisting of:
(i) Stem Pro® hESC SFM (Life Technologies)
(ii) MTeSR® or MTeSR 2 (Stem Cell Technologies)
(iii) Stemline Pluripro® (Sigma)
(iv) Essential 6 (E6) or Essential 8 (E8) (Life Technologies or Stem Cell Technologies)
(v) Pluripro® (Cambridge Biosciences)
(vi) STEMium® (ScienCell)
(vii) Nutristem (Stemgent/Miltenyi)
(viii) Other custom or “home-made” medium, for example those based on IMDM, DMEM or DMEM/F12+ and that might include serum, serum replacement and/or be conditioned by MEF or other feeder/auxiliary cells.

Suitable base media may contain serum, serum replacement or be serum free.

A (stem) cell culture medium of this invention may comprise a base medium (for example any of those listed above) and one or more of the pro-survival compounds described herein. A stem cell culture medium of this invention may further include one or more other factors, for example growth, maintenance or differentiation factors, including (but not limited to) one or more selected from:
(i) a GSK3 inhibitor;
(ii) Bone Morphogenic Protein 4 (BMP4);
(iii) Vascular Endothelial Growth Factor (VEGF);
(iv) Wnt3A and/or Wnt5a;
(v) ActivinA;
(vi) Fibroblast Growth Factor α (FGFα);
(vii) Stem Cell Factor (SCF);
(viii) β-estradiol;
(ix) a phosphodiesterase inhibitor;
(x) Vascular Endothelial Growth Factor 165 (VEGF);
(xi) Insulin-like Growth Factor 2 (IGF2);
(xii) Thrombopoietin (TPO);
(xiii) Heparin;
(xiv) Hydrocortisone;
(xv) Flt3-Ligand;
(xvi) Interleukin 3 (IL3);
(xvii) Interleukin (IL11);
(xviii) Erythropoietin (EPO);
(xix) Insulin Growth Factor 1 (IGF1);
(xx) StemRegenin1 (SR1);
(xxi) Pluripotin (SC1); and
(xxii) basic Fibroblast Growth Factor (bFGF)

One of skill will appreciate that depending on the stem cell to be cultured and/or any intended differentiation pathway, the type, number and amount of any growth or differentiation factor to be added may vary.

In a fourth aspect, the invention provides a method of maintaining stem cells in culture, said method comprising contacting a stem cell with one or more compounds according to formula (I), (II), (III) or (IV).

It should be understood that the term “maintain” may be applied to cells which, during all or part of the culture period and/or over a number of passages, remain viable, multipotent and/or retain normal karyotypic stability. A stem cell subjected to a method, of this invention may be maintained in culture for longer than a stem cell which has not been subjected to a method, of this invention.

The method may comprise contacting stem cells with a cell culture medium according to the third aspect of this invention. The method may comprise supplementing a stem cell medium with one or more of the pro-survival compounds described herein prior to seeding with stem cells and/or prior to a step of passaging the cells. Alternatively, or additionally, one or more of the pro-survival compounds described herein may be added to a stem cell medium intermittently or continuously during the culture and/or growth of stem cells.

The method may be applied to any of the stem cells described herein. Furthermore, the method may be used to maintain mechanically and/or chemically dissociated stem cells. For example, a method according to the fourth aspect of this invention may be applied to enzymatically dissociated stem cells.

A method according to the fourth aspect of this invention may be used to promote the survival of stem cells before, during and/or after passage, wherein through contact with a compound according to formula (I), (II), (III) or (IV), the stem cell retains one or more of the following properties:
(i) viability
(ii) a multipotent phenotype; and/or
(iii) normal karyotypic stability.

As stated, the pro-survival compounds of this invention may be used to promote or facilitate the survival of stem cells as they are passaged. For example, stem cells to be passaged and/or a stem cell medium may be contacted/supplemented with a pro-survival compound of this invention. For example, stem cells to be passaged and/or a stem cell medium may be contacted/supplemented with a pro-survival compound of this invention at any suitable time; for example at any time between about 1 minute and about 36 hours prior to a passage event and/or other event which might affect cell viability and/or survival (for example a media change event). For example, a pro-survival compound may be used from about 15 or 30 minutes prior to a passage event. The pro-survival compounds may be used 1 hour, 2 hours, 4 hours, 8 hours, 12 hours or 24 hours prior to a passage event.

Stem cells to be passaged or a stem cell medium may be contacted/supplemented with a pro-survival compound of this invention when the stem cells to be passaged exhibit some predetermined degree of confluence (e.g. 50-95%, 60-90% or 70-80% confluence). At the point of passage, the medium may be removed and/or the cells may be washed. Adherent cells may then be removed and/or the cells dissociated by any suitable means including, for example the use of an enzyme such as trypsin. The cells may then be harvested and/or washed and then re-plated at the required density. All further passages may be performed using this protocol where the supplementation of the medium with a pro-survival compound of this invention prior to passage, facilitates and/or promotes stem cell survival. Alternatively, rather than adding the pro-survival compound prior to passage, the compound may be continuously added to the cell culture medium.

In view of the above, the various compounds, compositions and methods described herein may find application in any stem cell protocols which require the maintenance and/or passage of stem cells over prolonged periods of time. Moreover, the various compounds, compositions and methods described herein may be used in protocols which affect the differentiation of embryoid bodies, single stem cells, stem cell monolayers and/or stem cells cultured and/or seeded onto carriers (for example cell scaffolds and the like).

PCT/GB2013/051917 (the entire contents of which are incorporated herein by reference) describes a protocol which may be used to induce and support the differentiation of stem cells into erythroid cells. Any one or more of the compounds described herein may be used in this protocol to facilitate or promote the survival (i.e. maintenance of viability and multipotency) of the stem cells as they are cultured, dissociated and/or passaged. One or more of the protocol steps described in PCT/GB2013/051917 may be supplemented with a compound of this invention. Alternatively, a compound of this invention may be used throughout the entire protocol. A compound of this invention may be used in the protocols of PCT/GB2013/051917 in order to promote stem cell survival as cells are passaged and/or dissociated from culture before the induction of differentiation.

The methods of this invention not only represent an improvement over methods which lack the use of any pro-survival compounds, but are comparable (in terms of stem cell survival effect and maintenance of multipotency) to methods based upon prior art pro-survival compounds such as, for example Y27632. Moreover, since the compounds of this invention are ROCK independent pro-survival compounds, the methods described herein are not associated with some of the deficiencies of prior art ROCK inhibition based methods which may, for example, result in stem cell karyotypic instability.

The compounds and methods of this invention may be used to support the chemical, mechanical and/or enzymatic passaging, disaggregating and/or dissociation of stem cells, including hPSCs. The inventors have noted that stem cells maintained in the presence of one or more of the compounds described herein may be passaged and/or dissagregated/dissociated a number of times. For example, the methods of this invention facilitate at least, for example 1, 5, 10, 15, 20, 25, 30, 35 or more stem cell passages and/or phases of disaggregation/dissociation.

In a fifth aspect, the invention provides the use of a compound according to formula (I), (II), (III) or (IV) in the culture of stem cells, wherein the use of a compound according to formula (I), (II), (III) or (IV) improves or promotes the survival and/or maintenance of stem cells in culture.

In a sixth aspect, the invention provides a kit for maintaining stem cells in culture and/or for use in a method of maintaining stem cells in culture as described herein, said kit comprising one or more of the pro-survival compounds described herein and/or a cell culture media according to the second aspect of this invention and one or more further components selected from the group consisting of:

(a) receptacles for the culture and/or maintenance of stem cells, embryoid bodies and/or cells;

(b) tools and/or implements for adding supplements to media (for example pipettes and/or syringes); and

(c) instructions for use.

For example, the receptacles for use may be sterile and may take the form of culture flasks and the like. The receptacles may comprise textured and/or profiled surfaces. The kits may further comprise cell culture scaffolds or matricies.

In a seventh aspect, the present invention provides a cell, for example a stem cell or a differentiated stem cell produced or obtainable by or using any one of the methods described herein. This aspect of the invention further relates to an erythroid cell produced or obtainable by or using any one of the methods described herein.

In an eighth aspect the present invention provides a method for the preparation of a compound according to formula (I), (II), (III) or (IV).

In some embodiments, a method for the preparation of a compound according to formula (I), (II), (III) or (IV) may comprise reacting a compound according to formula (V) with a species according to formula (VI) in the presence of a base. Such a method is illustrated in scheme 1 below.

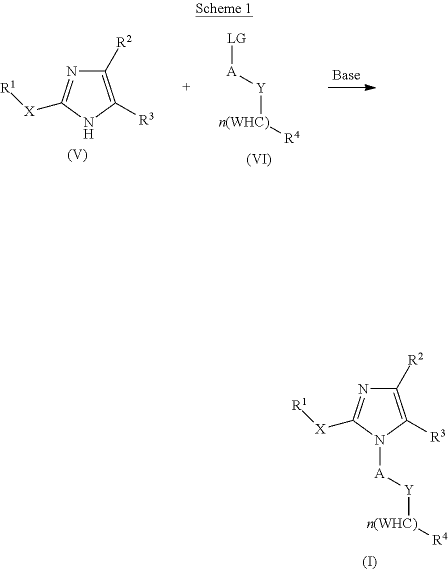

In the above reaction scheme, R¹, R², R³, X, A, Y, W, n and R⁴ are as defined for formula (I).

The group LG may be a leaving group. The leaving group may be displaced from formula (VI) during the alkylation reaction. Representative examples of leaving groups may include, but are not limited to, halo groups (e.g. chloro or bromo) and tosylate groups.

The base may act to deprotonate the compound according to formula (V). In some embodiments, the base may be sodium hydride.

In some embodiments, the reaction may take place in the presence of a solvent. A representative example of a suitable solvent includes, but is not limited to, N,N-dimethylformamide (DMF).

In a ninth aspect, the present invention provides methods and media as described above and in the detailed description and figures which follow.

### DETAILED DESCRIPTION

Note: compound SC332 (also referred to as “T16”) is 2-(2-(benzylamino)-1H-benzo[d]imidazol-1-yl)-N-(5-methylisoxazol-3-yl)acetamide and its structure is illustrated in the following Examples section).

The present invention will now be described by way of example only, and with reference to the following Figures, wherein:

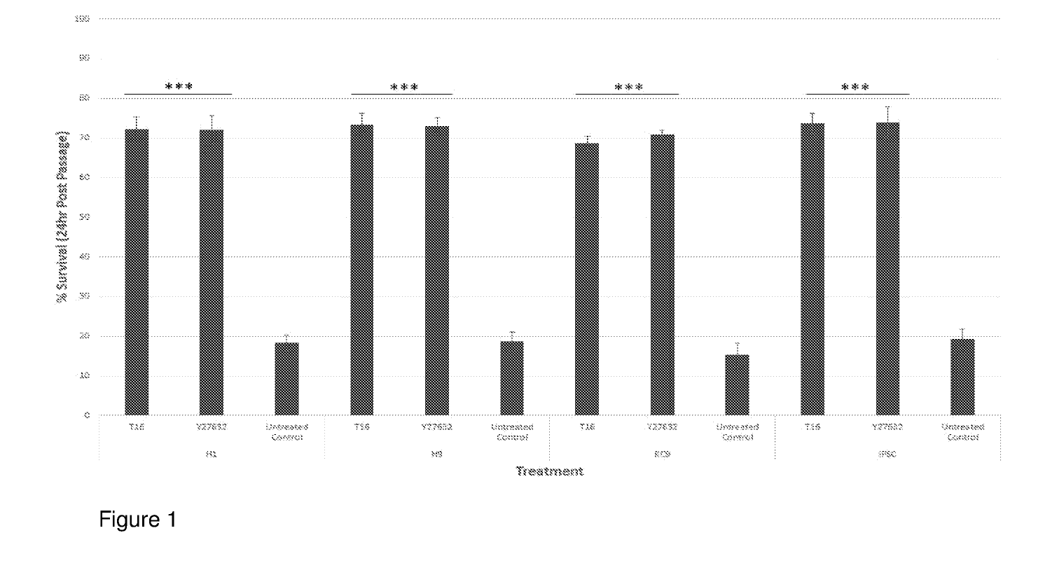

FIG. 1 shows the percentage survival (24 hour post passage) of cell lines treated with either 2-(2-(benzylamino)-1H-benzo[d]imidazol-1-yl)-N-(5-methylisoxazol-3-yl)acetamide (SC332), a representative compound of the present invention, or Y27632, or left untreated.

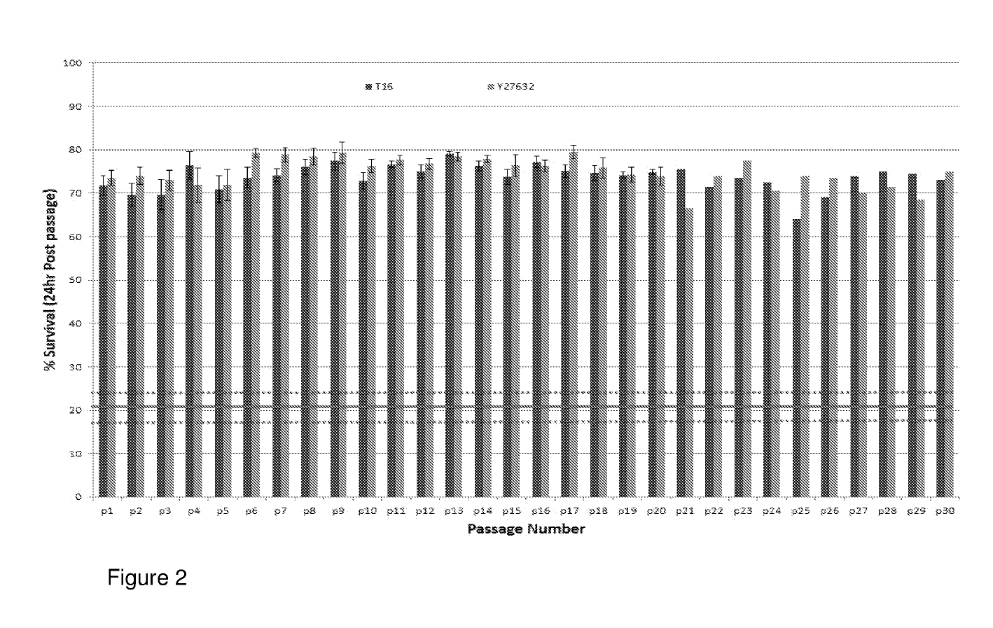

FIG. 2 shows hPSC survival after long term exposure to pro-survival compounds. SC332 or Y27632 was used to enzymatically passage the hESC line H1 for 30 consecutive passages. Survival was assayed 24 hrs post passage. Data up to passage 20 represents mean survival ±SEM, n=3. Data after passage 20 represents cell survival, n=1. The horizontal red line represents the average survival of untreated cells ±SEM, n=3.

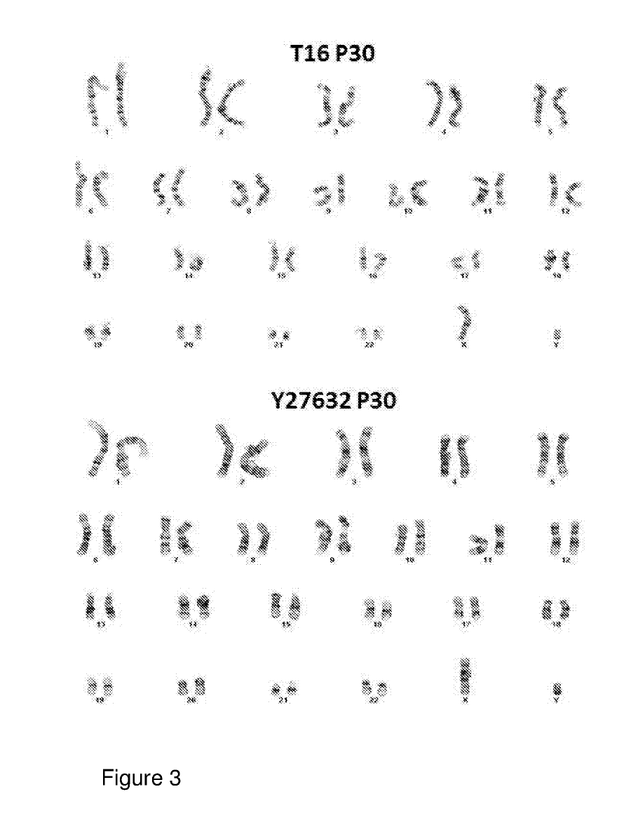

FIG. 3 shows the cytogenetic stability of cells treated with compound SC332. hPSC (H1 hESC) treated with SC332 or Y27632 for 30 consecutive passages were independently tested for cytogenetic stability (Yorkhill hospital, NHS Greater Glasgow and Clyde). Cells remained karyotypically normal (46, XY) until at least P30.

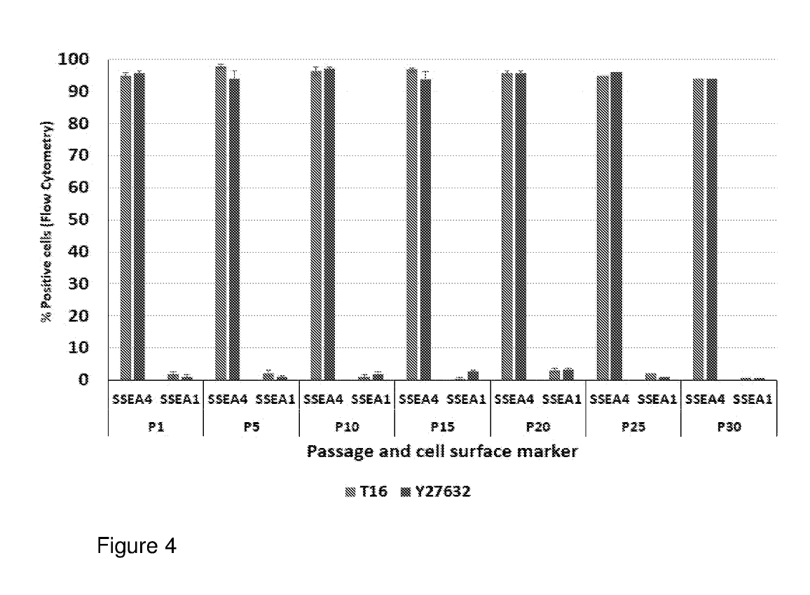

FIG. 4 shows flow cytometric analysis of hPSC surface markers. HPSC (H1 hESC) were enzymatically passaged using SC332 or Y27632 for 30 consecutive passages. Cells were harvested at the point of passage and analysed via flow cytometry for the pluripotency marker SSEA4 and differentiation marker SSEA1. The data shows that both SC332 and Y27632 treated cells maintained consistently high expression of SSEA4 over 30 consecutive passages, with negligible expression of SSEA1 being observed. Data shown as mean percentage of positive cells ±SEM (n=3) up to and including passage 20. Passages 25 and 30 represent values from an n=1.

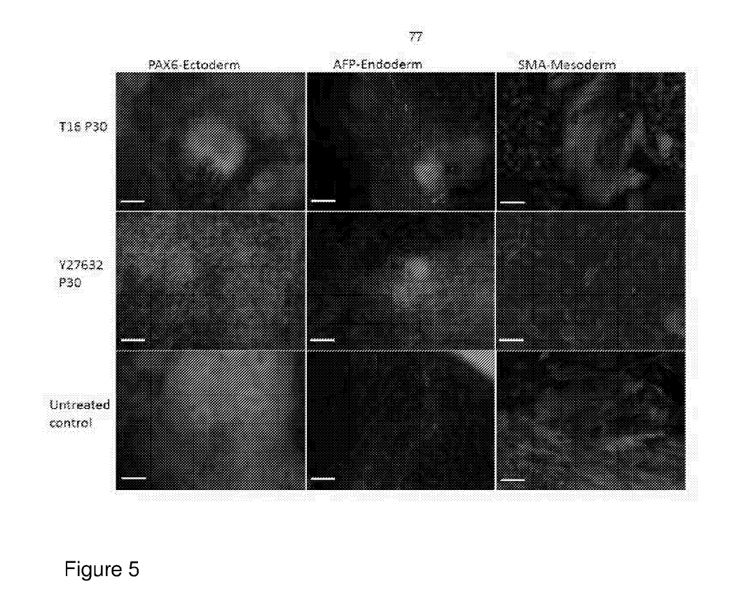

FIG. 5 shows passive differentiation of hPSC after treatment with compound SC332. HPSC treated for 30 passages with SC332 or Y27632 were passively differentiated for 2 weeks in suspension culture, followed by 2 weeks adherent culture, fixed and stained for markers of the three germ layers. Control cells that had been mechanically passaged and unexposed to any survival compound were also differentiated and stained in the same way. PAX6 is stained in the left hand column, AFP in the centre column and SMA in the right hand column. All cells were co-stained with DAPI. Scale bar represents 100 μm.

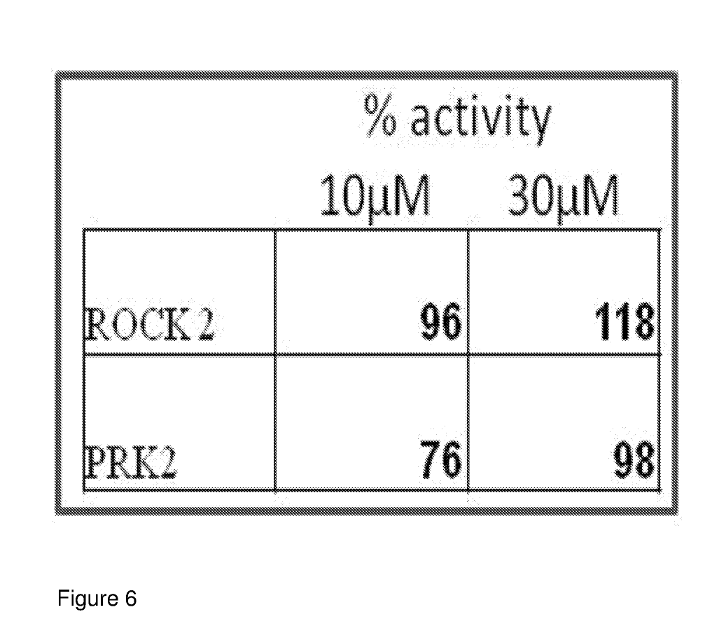

FIG. 6 shows the effect of compound SC332 on the activity of Rho-associated protein kinase 2 (ROCK2), evaluated at 10 μM and 30 μM concentrations using a radioactive filter-binding assay at the Dundee International Centre for Kinase Profiling. A 30 μM concentration of compound SC332 did not inhibit either ROCK2 or the closely related kinase, Serine/threonine-protein kinase N2 (PRK2).

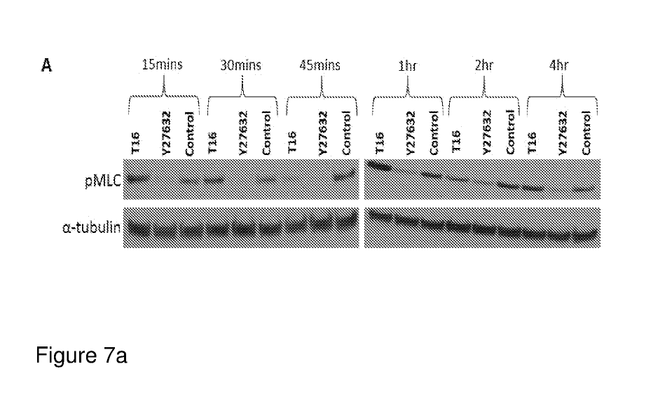

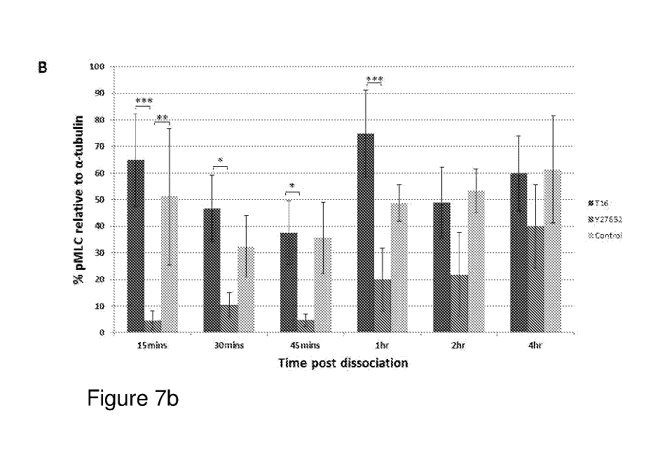

FIG. 7 shows analysis of levels of phosphorylated myosin light chain (pMLC) after treatment and dissociation of hPSC. Protein samples were taken from iPSC (cell line NMF-iPS6) that were treated with SC332, Y27632 or had no treatment (control) before being dissociated. Samples were harvested 15 mins, 30 mins, 45 mins, 1 hr, 2 hr and 4 hr post dissociation. Western blot analysis was performed. (A) shows representative immunoblots for pMLC and α-tubulin. (B) shows expression of pMLC relative to α-tubulin analysed via densitometry. Both SC332 treated and untreated cells had significantly higher levels of pMLC 15 mins post dissociation when compared to Y27632 treated cells (P=<0.001 and P=<0.01 respectively). SC332 treated cells had significantly higher levels of pMLC when compared to Y27632 treated cells after 30 mins (P=<0.05), 45 mins (P=<0.05) and 1 hr (P=<0.001). Data shown as mean±SEM, n=3.

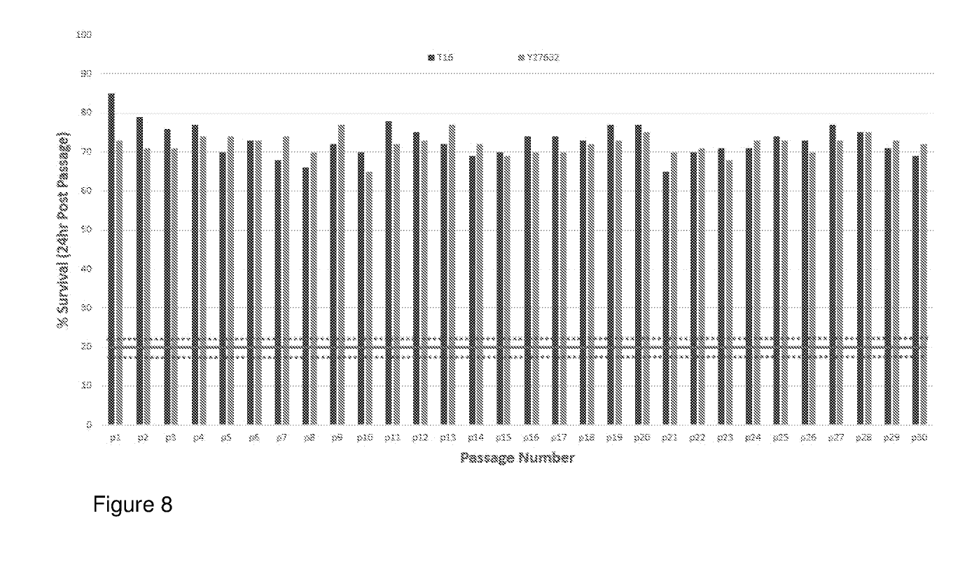

FIG. 8 shows the long term survival produced using human induced pluripotent stem cells (hiPSC). Cells (NMF-iPS6) were treated with either SC332 or Y27632 and passaged enzymatically for 30 consecutive passages. Data represents survival from a single experiment (n=1). The red line represents the average survival of untreated cells ±SEM, n=3.

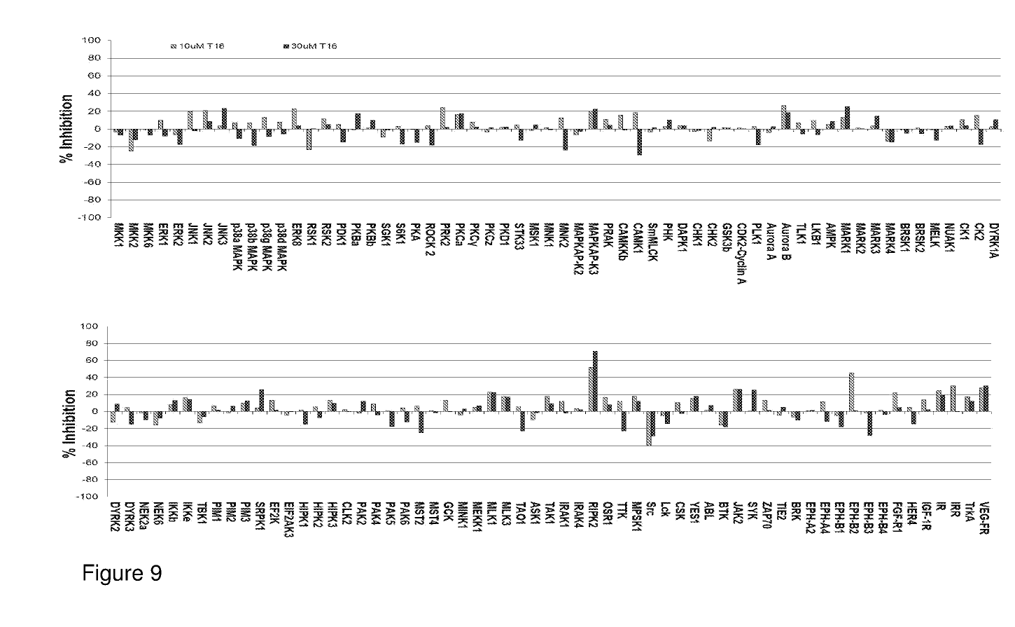

FIG. 9: Kinase profile of SC332 at 10 μM and 30 μM. Kinase inhibition on 121 kinases was assayed using a radioactive filter-binding assay at the Dundee International Centre for Kinase Profiling.

### EXAMPLES SECTION

Chemistry

As used in the following example reaction schemes, R¹, X, R² and R³ simply represent variable groups and have been used for illustrative purposes throughout each reaction scheme. The appropriate selection of these groups is dependent upon the targeted compound and will be apparent to the skilled person. They may not necessarily be defined as described in the foregoing.

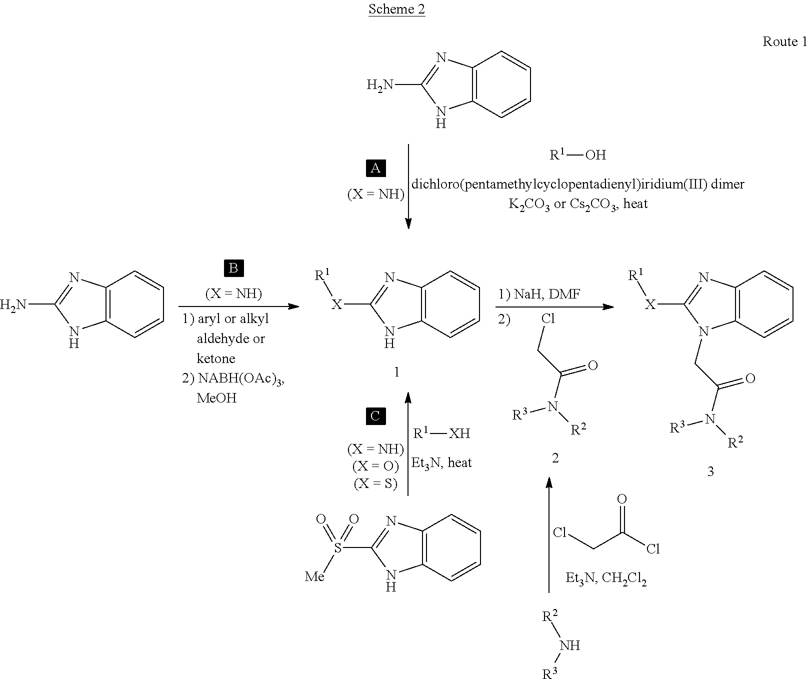

Compounds of formula (I) in which A is CH₂ and Y is simultaneously C(═O)NZ′ may be prepared as illustrated in Scheme 2, wherein a 2-substituted benzimidazole (1; X═O, S, NH) is alkylated with a chloroacetamide derivative (2) under basic conditions, typically by addition of sodium hydride to a solution of the benzimidazole (1) in anhydrous N,N-dimethylformamide solvent followed by addition of the chloroacetamide (2). The chloroacetamide (2) may be prepared by reaction of a suitable amine (Scheme 2, R²—NH—R³) with chloroacetyl chloride in the presence of a base (typically triethylamine) in an aprotic anhydrous solvent such as dichloromethane. Bromo- or iodoacetamide analogs of chloroacetamide 2 may alternatively be used. The benzimidazole precursor (1) may be commercially available or prepared by general methods known and widely used in the art such as: (A) N-alkylation of 2-aminobenzimidazole with an alcohol catalysed by dichloro(pentamethylcyclopentadienyl)iridium(III) dimer (F. Li et al., Eur. J. Org. Chem., 2012, 5085-5092); (B) reductive amination of 2-aminobenzimidazole, typically through condensation with an aldehyde or ketone and treatment of the intermediate imine with a reducing agent such as sodium triacetoxyborohydride in an appropriate solvent; (C) reaction of 2-(methylsulfonyl)-1H-benzo[d]imidazole with a sulfur, oxygen or nitrogen nucleophile (P. Lan et al., Tetrahedron Lett., 2008, 49, 1910-1914).

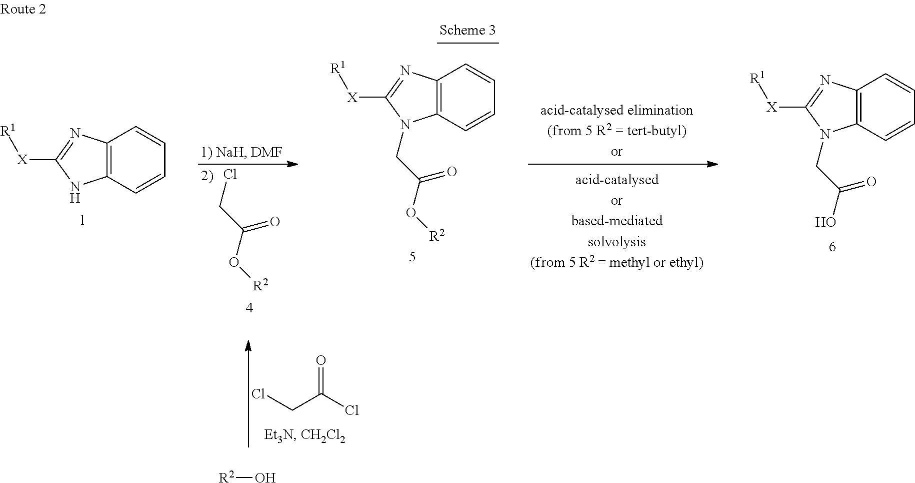

Compounds of formula (I) in which A is CH₂ and Y is simultaneously C(═O)O may be prepared as illustrated in Scheme 3, wherein a 2-substituted benzimidazole (1; X═O, S, NH) is alkylated with a chloroacetate ester (4) under basic conditions, typically by addition of sodium hydride to a solution of the benzimidazole (1) in anhydrous N,N-dimethylformamide solvent followed by addition of the chloroacetate ester (4). The chloroacetate (4) may be commercially available (e.g. R²=methyl, ethyl, tert-butyl) or prepared by reaction of a suitable alcohol (Scheme 3, R²—OH) with chloroacetyl chloride in the presence of a base (typically triethylamine) in an aprotic anhydrous solvent such as dichloromethane. The benzimidazole precursor (1) may be commercially available or prepared as presented in Route 1. Acid-catalysed or base-mediated solvolysis of esters 5 may afford access to the corresponding carboxylic acids 6. Alternatively an acid-catalysed elimination reaction on 5 (R²=tert-butyl), typically accomplished by treatment with trifluoroacetic acid or a mixture of trifluoroacetic acid in dichloromethane, may afford carboxylic acids 6.

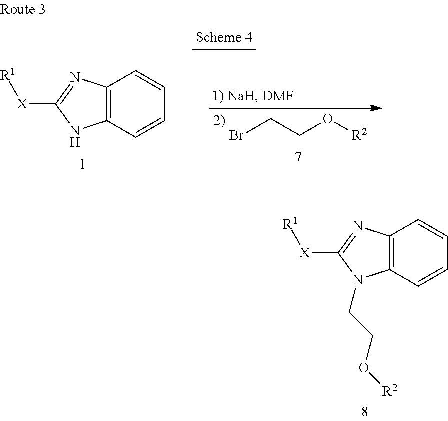

Compounds of formula (I) in which A is CH₂CH₂ and Y is simultaneously 0 may be prepared as illustrated in Scheme 4, wherein a 2-substituted benzimidazole (1; X═O, S, NH) is alkylated with a bromoethyl ether (4) under basic conditions, typically by addition of sodium hydride to a solution of the benzimidazole (1) in anhydrous N,N-dimethylformamide solvent followed by addition of the bromoethyl ether (4). Chloro- or iodoethyl ether analogs of 4 may be used or the corresponding toluenesulfonates or trifluoromethanesulfonates. The benzimidazole precursor (1) may be commercially available or prepared as presented in Route 1.

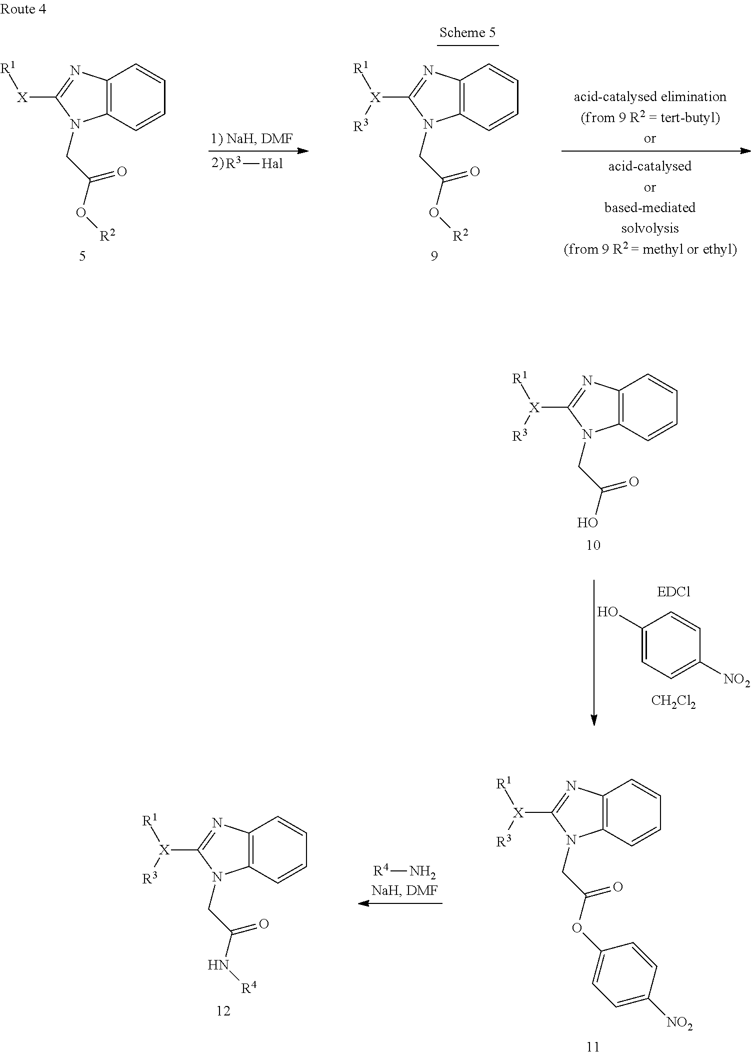

Compounds of formula (I) in which A is CH₂ and Y is simultaneously C(═O)NZ′ and X is simultaneously N-alkyl may be prepared as illustrated in Scheme 5, wherein a benzimidazolylacetate derivative (5; X═NH) is alkylated under basic conditions, typically by addition of sodium hydride to a solution of 5 in anhydrous N,N-dimethylformamide solvent followed by addition of an alkyl halide (Scheme 5 R³-Hal). Conversion of esters 9 into acids 10 may be accomplished by acid-catalysed or base-mediated solvolysis of the ester or alternatively by an acid-catalysed elimination reaction on 9 (R²=tert-butyl), typically through treatment with trifluoroacetic acid or a mixture of trifluoroacetic acid in dichloromethane. Acids 10 may be converted into their para-nitrophenol ester derivatives (11), for example by treatment with 1-ethyl-3-(3-dimethylaminopropyl)carbodiimide and para-nitrophenol in dichloromethane. Reaction of esters 11 with a suitable primary amine pre-treated with sodium hydride in N,N-dimethylformamide may then afford amides 12. Those skilled in the art will appreciate that numerous alternative reagents and conditions may be applied to activate carboxylic acids such as 11 for coupling to amines.

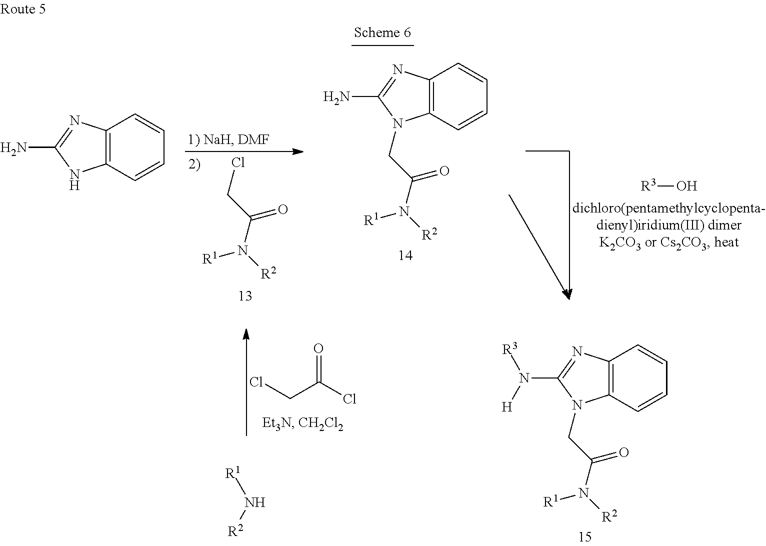

Compounds of formula (I) in which A is CH₂ and Y is simultaneously C(═O)NZ′ may also be prepared as illustrated in Scheme 6, wherein 2-aminobenzimidazole is alkylated with a chloroacetamide derivative (13) under basic conditions, typically by addition of sodium hydride to a solution of 2-aminobenzimidazole in anhydrous N,N-dimethylformamide solvent followed by 13. The chloroacetamide (13) may be prepared by reaction of a suitable amine (Scheme 6, R¹—NH—R²) with chloroacetyl chloride in the presence of a base (typically triethylamine) in an aprotic anhydrous solvent such as dichloromethane. Bromo- or iodoacetamide analogs of chloroacetamide 13 may alternatively be used. Substituted benzimidazoles 14 may then be converted into targets 15 by reaction with suitable alcohols (R³OH) catalysed by dichloro(pentamethylcyclopentadienyl)iridium(III) dimer.

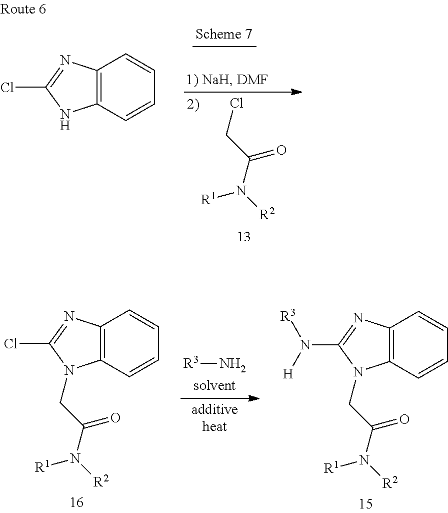

Compounds of formula (I) in which A is CH₂ and Y is simultaneously C(═O)NZ′ may additionally be prepared as illustrated in Scheme 7, wherein 2-chlorobenzimidazole is alkylated with a chloroacetamide derivative (13) under basic conditions, typically by addition of sodium hydride to a solution of 2-chlorobenzimidazole in anhydrous N,N-dimethylformamide solvent followed by 13. As previously, bromo- or iodoacetamide analogs of chloroacetamide 13 may alternatively be used. Substituted benzimidazoles 16 may then be converted into targets 15 by reaction with suitable amines (R³—NH₂), for example by heating in an appropriate solvent (e.g. 1,4-dioxane or n-butanol) in a sealed pressure vessel (if required) and sometimes in the presence of a suitable additive such as potassium dihydrogen phosphate or a silver salt.

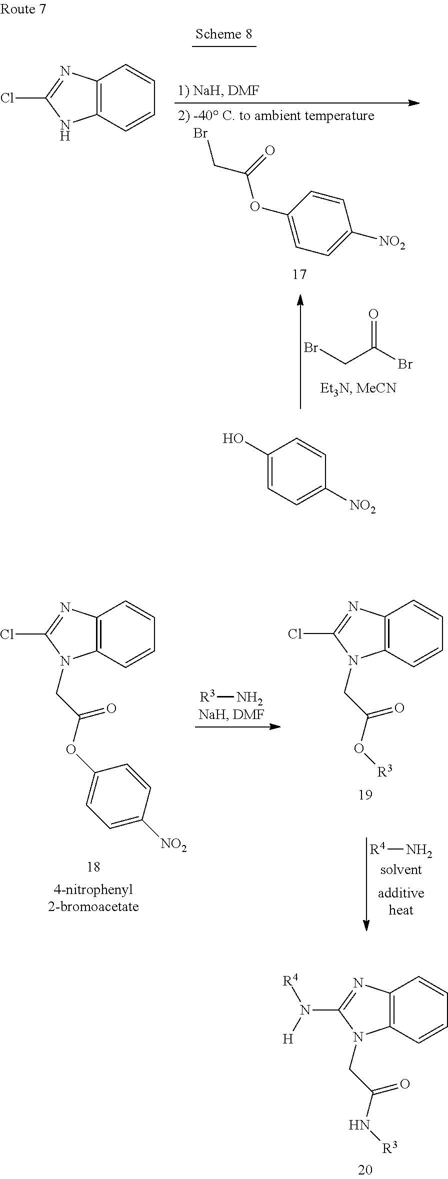

A variant on the preceding route to compounds of formula (I) in which A is CH₂ and Y is simultaneously C(═O)NZ′ is illustrated in Scheme 8, wherein 2-chlorobenzimidazole is alkylated with 4-nitrophenyl 2-bromoacetate (17), for example by addition of sodium hydride to a solution of 2-chlorobenzimidazole in anhydrous N,N-dimethylformamide solvent followed by 17. For this purpose bromoacetate 17 may first be prepared by reaction of bromoacetyl bromide with para-nitrophenol. Reaction of intermediate esters 18 with a suitable primary amine (R³—NH₂) pre-treated with sodium hydride in N,N-dimethylformamide may then afford amides 19. The latter may then be converted into targets 20 by a reaction with a suitable amine (R⁴—NH₂), for example by heating in an appropriate solvent (e.g. 1,4-dioxane or n-butanol) in a sealed pressure vessel (if required) and sometimes in the presence of a suitable additive such as potassium dihydrogen phosphate or a silver salt.

General Chemistry Experimental

Tetrahydrofuran (THF), diethyl ether (Et₂O), dichloromethane (DCM) and toluene (PhMe) were dried by passage through commercial columns in an in-house solvent purification system. Methanol (MeOH), ethyl acetate (EtOAc), chloroform, acetone, hexane and light petroleum were used as supplied from Fisher. ‘Light petroleum’ refers to the fraction boiling between 40° C. and 60° C. Anhydrous N,N-dimethylformamide (DMF) was purchased from Aldrich and used as supplied from Sure/Seal™ bottles. Analytical thin layer chromatography (TLC) was carried out using aluminium backed plates coated with Merck Kieselgel 60 GF254 (Art. 05554). Developed plates were visualized under ultra-violet light (254 nm) and/or alkaline potassium permanganate dip. Preparative chromatography was performed using flash silica (60 Å; 35-70 μM) from Fisher in glass columns or over Strata® SI-1 (70 Å; 55 μM) silica Giga™ Tube cartridges from Phenomenex.

IR spectra were recorded on a Thermo Scientific iD5 Diamond ATR/Nicolet iS5 FT-IR spectrometer with samples as neat solids or liquids. Mass spectra were obtained under electrospray ionisation (ESI) conditions through the Edinburgh University Mass Spectrometry Service. ¹H NMR spectra were recorded at 300 and 400 MHz on Bruker AVIII-300 and AVIII-400 spectrometers; ¹³C NMR spectra were recorded at 75 and 101 MHz on the same instruments. Chemical shifts are recorded in parts per million (8 in ppm) and are referenced against solvent signals (δ_{C} 77.16 for chloroform, δ_{C} 39.52 for methyl sulfoxide) for ¹³C spectra and solvent residual resonances (δ_{H} 7.26 for chloroform, δ_{H} 3.31 for methanol, δ_{H} 2.50 methyl sulfoxide) for ¹H spectra. Chemical shift values and are accurate to ±0.01 ppm and ±0.1 ppm in ¹H and ¹³C spectra respectively. J values are given in Hz. Multiplicity designations used are: s, d, t, q, sept and m for singlet, doublet, triplet, quartet, septet and multiplet respectively; broadened signals are denoted br. In ¹³C NMR spectra, signals corresponding to CH, CH₂, or CH₃ groups are assigned from DEPT. Elemental analyses were carried out by the analytical service of the at Heriot-Watt University using an Exeter CE-440 Elemental Analyser.

### Example 1

### 2-(2-(Benzylamino)-1H-benzo[d]imidazol-1-yl)-N-(2-methoxyethyl)acetamide (SC050) was prepared according to Route 1

Step 1. N-Benzyl-1H-benzo[d]imidazol-2-amine was prepared following a published procedure (F. Li et al., Eur. J. Org. Chem., 2012, 5085-5092). Thus, a flame-dried, heavy-walled, sealable flask fitted with a magnetic stir bar was charged under argon with 2-aminobenzimidazole (1.00 g, 7.51 mmol), dichloro(pentamethylcyclopentadienyl)iridium(III) dimer (12.0 mg, 15.1 μmol), cesium carbonate (245 mg, 752 μmol) and benzyl alcohol (3.11 mL, 30.1 mmol). The flask was sealed and the mixture heated at 130° C. with stirring. After 24 h the mixture was cooled, diluted with EtOAc (60 mL) and washed with brine (3×20 mL). The organic phase was dried (MgSO₄), filtered and evaporated to afford a residual green oil that was subjected to silica gel chromatography (gradient elution with 90% v/v hexane/EtOAc to 100% EtOAc). Fractions containing the target material (R_{f} 0.32, 10% v/v MeOH/DCM) were combined and evaporated to afford N-benzyl-1H-benzo[d]imidazol-2-amine (1.52 g, 6.79 mmol; 90%) as a light brown powder: δ_{H} (300 MHz, CD₃OD) 7.38-7.42 (2H, m), 7.30-7.36 (2H, m), 7.17-7.28 (3H, m), 6.94-7.00 (2H, m), 4.59 (2H, s).

Step 2. To an ice-cooled solution of 2-methoxyethylamine (623 mg, 8.29 mmol) and triethylamine (1.50 mL, 10.8 mmol) in anhydrous DCM (50 mL) under argon was added dropwise chloroacetyl chloride (795 μL, 10.0 mmol). After 4 h the mixture was flushed through a Strata® SI-1 silica cartridge (20 g Giga™ Tube), eluting with DCM. Fractions containing the target material (R_{f} 0.65, 100% EtOAc) were combined and evaporated to afford 2-chloro-N-(2-methoxyethyl)acetamide (1.14 g, 7.50 mmol; 90%) as a brown oil: δ_{H} (300 MHz, CDCl₃): 6.92 (1H, br s), 4.02 (2H, s), 3.44-3.49 (4H, m), 3.34 (3H, s).

Step 3. To an ice-cooled solution of N-benzyl-1H-benzo[d]imidazol-2-amine (224 mg, 1.00 mmol) in anhydrous DMF (5 mL) under argon was added NaH (60% w/w dispersion in mineral oil; 44.0 mg, 1.10 mmol). After 20 min a solution of 2-chloro-N-(2-methoxyethyl)acetamide (194 mg, 1.28 mmol) in DMF (5 mL) was added. The mixture was stirred and allowed to come ambient temperature over the course of 1 h. TLC analysis (100% EtOAc) indicated complete consumption of the benzimidazole substrate (R_{f} 0.30) and formation of a product (R_{f} 0.37). The reaction mixture was evaporated to dryness (70 CC, 12 mbar) and the resulting residue diluted with EtOAc (50 mL). This solution was then washed with brine (3×20 mL), dried (Na₂SO₄), filtered and evaporated to afford a solid residue. Recrystallization of the solid residue from hot CHCl₃/hexane afforded 2-(2-(benzylamino)-1H-benzo[d]imidazol-1-yl)-N-(2-methoxyethyl)acetamide (285 mg, 842 μmol; 84%) as a colourless powder: δ_{H} (300 MHz, CDCl₃) 7.46-7.49 (1H, ddd, J 7.8, 1.0 and 0.6), 7.36-7.40 (2H, m), 7.23-7.35 (3H, m), 7.12-7.17 (1H, m), 7.06 (1H, td, J 7.4 and 1.2), 7.01 (1H, ddd, J 7.8, 1.6 and 0.6), 6.51 (1H, br s), 5.65 (1H, br t, J 5.5), 4.67 (2H, d, J 5.3), 4.47 (2H, s), 3.27-3.33 (4H, m), 3.19 (3H, s).

### Example 2

### 2-(2-(Benzylamino)-1H-benzo[d]imidazol-1-yl)-N-(2-hydroxyethyl)acetamide (SC705) was prepared according to Route 1

Step 1. A flame-dried flask fitted with a magnetic stir bar was charged under argon with anhydrous THF (15 mL) and NaH (60% w/w dispersion in mineral oil; 110 mg, 2.75 mmol). To the resulting suspension was added ethanolamine (76 μL, 1.3 mmol) followed after 10 min by tert-butyldimethylsilyl chloride (192 mg, 1.27 mmol). After stirring at ambient temperature for 1 h chloroacetyl chloride (100 μL, 1.26 mmol) was added and stirring continued for a further 1.5 h to afford ‘Mixture A’ as a colourless liquid containing a fine solid suspension. In parallel with the preparation of Mixture A, a separate flask was charged with N-benzyl-1H-benzo[d]imidazol-2-amine (283 mg, 1.27 mmol), anhydrous THF (35 mL) and NaH (60% w/w dispersion in mineral oil; 60.0 mg, 1.50 mmol), generating a homogenous yellow solution as ‘Mixture B’ after stirring for 30 min. Mixture A was then cannulated into Mixture B and stirring continued. After 24 h the combined mixture was filtered through Celite® and the filtrate evaporated to dryness. The resulting residue was subjected to chromatography over a Strata® SI-1 silica cartridge (20 g Giga™ Tube) using gradient elution: 100% light petroleum >25% v/v DCM/light petroleum >75% v/v DCM/light petroleum >100% DCM >10% v/v EtOAc/DCM >20% v/v EtOAc/DCM >30% v/v EtOAc/DCM >40% v/v EtOAc/DCM. Fractions containing the product, which eluted in 30-40% v/v EtOAc/DCM, were combined and evaporated to afford 2-(2-(benzylamino)-1H-benzo[d]imidazol-1-yl)-N-(2-((tert-butyldimethylsilyl)oxy)ethyl)acetamide (274 mg, 625 μmol; 50%) as a colourless powder: δ_{H} (300 MHz, CDCl₃) 7.52 (1H, br d, J 7.7), 7.27-7.42 (5H, m), 7.16 (1H, td, J 7.4 and 1.5), 7.08 (1H, td, J 7.4 and 1.1), 7.03 (1H, br dd, J 7.8 and 1.0), 6.11 (1H, br t, J 4.7), 5.04 (1H, br t, J 5.3), 4.73 (2H, d, J 5.5), 4.51 (2H, s), 3.57 (2H, t, J 5.3), 3.31 (2H, q, J 5.3), 0.75 (9H, s), −0.08 (6H, s).

Step 2. To a stirred solution of 2-(2-(benzylamino)-1H-benzo[d]imidazol-1-yl)-N-(2-((tert-butyldimethylsilyl)oxy)ethyl)acetamide (274 mg, 625 μmol) in THF (15 mL) was added tetrabutylammonium fluoride (1 M solution in THF containing 5% water; 750 μL, 750 μmol). After 24 h the mixture was evaporated to dryness, affording a viscous yellow oil that was subjected to chromatography over a Strata® SI-1 silica cartridge (20 g Giga™ Tube) using gradient elution: 100% DCM >20% v/v EtOAc/DCM >40% v/v EtOAc/DCM >5% v/v MeOH/DCM. Fractions containing the product, which eluted in 5% v/v MeOH/DCM, were combined and evaporated to afford 2-(2-(benzylamino)-1H-benzo[d]imidazol-1-yl)-N-(2-hydroxyethyl)acetamide (175 mg, 539 μmol; 86%) as a colourless powder: δ_{H} (300 MHz, CD₃OD) 7.39-7.43 (2H, m), 7.27-7.34 (3H, m), 7.20-7.25 (1H, m), 6.97-7.10 (3H, m), 4.70 (2H, s), 4.66 (2H, s), 3.60 (2H, t, J 5.7), 3.34 (2H, t, J 5.7).

### Example 3

### tert-Butyl 2-(2-(benzylamino)-1H-benzo[d]imidazol-1-yl)acetate (SC343) was prepared according to Route 2

To an ice-cooled solution of N-benzyl-1H-benzo[d]imidazol-2-amine (224 mg, 1.00 mmol) in anhydrous DMF (5 mL) under argon was added NaH (60% w/w dispersion in mineral oil; 44.0 mg, 1.10 mmol). After 20 min a solution of tert-butyl bromoacetate (218 mg, 1.12 mmol) in DMF (5 mL) was added. The mixture was stirred and allowed to come ambient temperature over the course of 1 h. TLC analysis (100% EtOAc) indicated complete consumption of the benzimidazole substrate (R_{f} 0.30) and formation of a product (R_{f} 0.83). The reaction mixture was evaporated to dryness (70° C., 12 mbar) and the resulting residue diluted with EtOAc (50 mL). This solution was then washed with brine (3×20 mL), dried (Na₂SO₄), filtered and evaporated to afford a viscous oil that was subjected to chromatography over a Strata® SI-1 silica cartridge (20 g Giga™ Tube), eluting with 100% DCM followed by 100% EtOAc. Fractions containing the product were combined and evaporated to afford tert-butyl 2-(2-(benzylamino)-1H-benzo[d]imidazol-1-yl)acetate (316 mg, 937 μmol; 94%) as a colourless oil that solidified on standing: δ_{H} (300 MHz, CDCl₃) 7.53 (1H, br d, J 7.4), 7.42-7.45 (2H, m), 7.27-7.39 (3H, m), 7.11-7.18 (1H, m), 7.06-7.09 (2H, m), 4.81 (1H, br t, J 5.2), 4.74 (2H, d, J 5.2), 4.50 (2H, s), 1.42 (9H, s).

### Example 4

### 2-(2-(Benzylamino)-1H-benzo[d]imidazol-1-yl)acetic acid (SC287) was prepared according to Route 2

To a stirred solution of tert-butyl 2-(2-(benzylamino)-1H-benzo[d]imidazol-1-yl)acetate (197 mg, 584 μmop in DCM (4 mL) was added trifluoroacetic acid (4 mL). After 4 h the mixture was evaporated to dryness and the resulting solid residue recrystallized from hot EtOAc to afford 2-(2-(benzylamino)-1H-benzo[d]imidazol-1-yl)acetic acid (110 mg, 391 μmol; 67%) as a colourless solid: δ_{H} (300 MHz, DMSO-d₆) 13.45 (1H, br s), 9.48 (1H, br s), 7.55-7.61 (1H, m), 7.25-7.44 (8H, m), 5.09 (2H, s), 4.71 (2H, d, J 5.8).

### Example 5

### N-Benzyl-1-(2-(benzyloxy)ethyl)-1H-benzo[d]imidazol-2-amine (SC175) was prepared according to Route 3

To an ice-cooled solution of N-benzyl-1H-benzo[d]imidazol-2-amine (225 mg, 1.01 mmol) in anhydrous DMF (5 mL) under argon was added NaH (60% w/w dispersion in mineral oil; 44.0 mg, 1.10 mmol). After 20 min a solution of (2-bromoethyl) benzyl ether (240 mg, 1.12 mmol) in DMF (5 mL) was added. The mixture was stirred and allowed to come ambient temperature over the course of 1 h. TLC analysis (100% EtOAc) indicated complete consumption of the benzimidazole substrate (R_{f} 0.30) and formation of a product (R_{f} 0.83). The reaction mixture was evaporated to dryness (70° C., 12 mbar) and the resulting residue diluted with EtOAc (50 mL). This solution was then washed with brine (3×20 mL), dried (Na₂SO₄), filtered and evaporated to afford a viscous oil that was subjected to chromatography over a Strata® SI-1 silica cartridge (20 g Giga™ Tube), eluting with 100% DCM followed by 100% EtOAc. Fractions containing the product were combined and evaporated to afford N-benzyl-1-(2-(benzyloxy)ethyl)-1H-benzo[d]imidazol-2-amine (308 mg, 861 μmol; 86%) as a yellow oil: δ_{H} (300 MHz, CDCl₃) 7.57 (1H, ddd, J 7.7, 1.2 and 0.6), 7.23-7.34 (8H, m), 7.14-7.20 (3H, m), 7.09 (1H, td, J 7.4 and 1.2), 7.04 (1H, ddd, J 7.7, 1.6 and 0.7), 5.69 (1H, br t, J 5.5), 4.63 (2H, d, J 5.6), 4.45 (2H, s), 4.12-4.17 (2H, m), 3.81-3.86 (2H, m).

### Example 6

### 2-(2-(Benzyl(methyl)amino)-1H-benzo[d]imidazol-1-yl)-N-(5-methylisoxazol-3-yl)acetamide (SC881) was prepared according to Route 4

Step 1. To an ice-cooled solution of tert-butyl 2-(2-(benzylamino)-1H-benzo[d]imidazol-1-yl)acetate (342 mg, 1.01 mmol) and iodomethane (124 μL, 1.99 mmol) in anhydrous DMF (5 mL) under argon was added NaH (60% w/w dispersion in mineral oil; 48.0 mg, 1.20 mmol). The reaction mixture was stirred and allowed to come to ambient temperature over the course of 48 h. The mixture was then evaporated to dryness (60° C., 12 mbar) and the resulting residue directly subjected to chromatography over a Strata® SI-1 silica cartridge (5 g Giga™ Tube), eluting with 100% DCM followed by 0.5% v/v MeOH/DCM. Fractions containing the product were combined and evaporated to afford tert-butyl 2-(2-(benzyl(methyl)amino)-1H-benzo[d]imidazol-1-yl)acetate (241 mg) as a yellow oil in ca. 90% purity by ¹H NMR analysis.

Step 2. The ester from Step 1 was taken up in DCM (6 mL), ice-cooled and treated with trifluoroacetic acid (2 mL). The mixture was then allowed to come to ambient temperature. After stirring for 16 h the mixture was evaporated to dryness, affording a residue that was subjected to chromatography over a Strata® SI-1 silica cartridge (5 g Giga™ Tube) using 1-10% v/v MeOH/DCM solvent gradient elution. Fractions containing the product were combined and evaporated to afford 2-(2-(benzyl(methyl)amino)-1H-benzo[d]imidazol-1-yl)acetic acid (150 mg, 508 μmol; 50% for Steps 1 and 2 combined) as a colourless solid.

Step 3. The acid from Step 2 (150 mg, 508 μmop was taken up in DCM (10 mL). para-Nitrophenol (88 mg, 630 μmol), N,N-dimethylaminopyridine (12 mg) and 1-ethyl-3-(3-dimethylaminopropyl)carbodiimide hydrochloride (146 mg, 762 μmol) were then added. After stirring for 1.5 h, the reaction mixture was diluted with DCM (30 mL), washed successively with water (2×10 mL) and brine (15 mL), dried (MgSO₄), filtered and evaporated. The resulting waxy solid (220 mg) was subjected to chromatography over a Strata® SI-1 silica cartridge (5 g Giga™ Tube), eluting with 100% DCM followed by 0.5% v/v MeOH/DCM. Fractions containing the product were combined and evaporated to afford 4-nitrophenyl 2-(2-(benzyl(methyl)amino)-1H-benzo[d]imidazol-1-yl)acetate (164 mg) as a 1:1 mixture with para-nitrophenol according to ¹H NMR analysis.

Step 4. The para-nitrophenol ester from Step 3 (ca. 1:1 mixture with para-nitrophenol; 162 mg, 0.29 mmol) was taken up in anhydrous DMF (2 mL) under argon and ice-cooled. A solution of 3-amino-5-methylisoxazole (44 mg, 0.45 mmol) in DMF (3 mL) that had been pretreated with NaH (60% w/w dispersion in mineral oil; 22 mg, 0.55 mmol) for 25 min was then added dropwise into the para-nitrophenol ester solution. The combined mixture was allowed to come to ambient temperature. After stirring for 16 h the reaction mixture was evaporated to dryness (60° C., 12 mbar) and the resulting residue reconstituted with CHCl₃ (30 mL). This solution was then washed successively with water (2×10 mL) and brine (15 mL), dried (MgSO₄), filtered and evaporated. The crude material (152 mg) thus obtained was subjected to chromatography over a Strata® SI-1 silica cartridge (5 g Giga™ Tube), eluting with 0.5% v/v MeOH/DCM followed by 1% v/v MeOH/DCM. Fractions containing the product were combined and evaporated. The resulting waxy solid residue was triturated with hexane/Et₂O to afford 2-(2-(benzyl(methyl)amino)-1H-benzo[d]imidazol-1-yl)-N-(5-methylisoxazol-3-yl)acetamide (16.1 mg, 43 μmol; 8% over Steps 3 and 4 combined) as a colourless powder: δ_{H} (300 MHz, CDCl₃) 8.31 (1H, br s), 7.65 (1H, ddd, J 7.7, 1.2 and 0.6), 7.24-7.35 (6H, m), 7.19 (1H, td, J 7.4 and 1.2), 7.14 (1H, ddd, J 7.7, 1.6 and 0.6), 6.70 (1H, br s), 4.80 (2H, s), 4.46 (2H, s), 2.91 (3H, s), 2.40 (3H, d, J 0.9).

### Example 7

### 2-(2-(Isobutylamino)-1H-benzo[d]imidazol-1-yl)-N-(2-methoxyethyl)acetamide (SC769) was prepared according to Route 5

Step 1. To a stirred solution of 1H-benzo[d]imidazol-2-amine (1.33 g, 10.0 mmol) in anhydrous DMF (20 mL) under argon was added NaH (60% w/w dispersion in mineral oil; 400 mg, 10.0 mmol) in portions at ambient temperature. After 10 min a solution of 2-chloro-N-(2-methoxyethyl)acetamide (preparation vide supra, EXAMPLE 1; 1.52 g, 10.0 mmol) in DMF (8 mL) was added. The mixture was then stirred at ambient temperature for 16 h. The reaction mixture was evaporated to dryness (60° C., 12 mbar) and the resulting residue subjected to chromatography over a Strata® SI-1 silica cartridge (20 g Giga™ Tube), eluting successively with 2%, 5% and 10% v/v MeOH/DCM. Fractions containing the product were combined and evaporated to afford 2-(2-amino-1H-benzo[d]imidazol-1-yl)-N-(2-methoxyethyl)acetamide (SC593, 925 mg, 3.72 mmol; 37%) as a buff powder: δ_{H} (300 MHz, CDCl₃) 7.46-7.50 (1H, m), 7.16-7.23 (1H, m), 7.08-7.14 (2H, m), 6.24 (1H, br s), 4.90 (2H, br s), 4.61 (2H, s), 3.42-3.53 (4H, m), 3.31 (3H, s).

Step 2. A flame-dried, heavy-walled, sealable tube fitted with a magnetic stir bar was charged under argon with 2-(2-amino-1H-benzo[d]imidazol-1-yl)-N-(2-methoxyethyl)acetamide (154 mg, 621 μmop, isobutanol (0.916 mL, 9.92 mmol), dichloro(pentamethylcyclopentadienyl)iridium(III) dimer (5 mg, 6 μmol) and cesium carbonate (20 mg, 62 μmol). The tube was sealed and the mixture heated at 130° C. with stirring. After 20 h the mixture was cooled and evaporated to afford a residue that was subjected to chromatography over a Strata® SI-1 silica cartridge (10 g Giga™ Tube), eluting successively with 10%, 20%, 50% and 75% v/v EtOAc/light petroleum followed by EtOAc. Fractions containing the product were combined and evaporated to afford 2-(2-(isobutylamino)-1H-benzo[d]imidazol-1-yl)-N-(2-methoxyethyl)acetamide (SC769) (88 mg, 0.29 mmol; 47%) as a colourless powder: δ_{H} (300 MHz, CDCl₃) 7.50-7.54 (1H, m), 7.14-7.20 (1H, m), 7.02-7.11 (2H, m), 6.32 (1H, br s), 5.07 (1H, br t, J 5.6), 4.54 (2H, s), 3.36-3.48 (6H, m), 3.27 (3H, s), 2.02 (1H, nonet, J 6.8), 1.04 (6H, d, J 6.7).

### Example 8

### N-(2-Methoxyethyl)-2-(2-((pyridin-2-ylmethyl)amino)-1H-benzo[d]imidazol-1-yl)acetamide (SC209) was prepared according to Route 6

Step 1. To a stirred, ice-cooled solution of 2-chloro-1H-benzo[d]imidazole (1.53 g, 10.0 mmol) in anhydrous DMF (20 mL) under argon was added NaH (60% w/w dispersion in mineral oil; 400 mg, 10.0 mmol) in portions. The mixture was removed from the ice bath, stirred at ambient temperature for 20 min and then returned to the ice bath prior to dropwise addition of a solution of 2-chloro-N-(2-methoxyethyl)acetamide (preparation vide supra, EXAMPLE 1; 1.52 g, 10.0 mmol) in DMF (8 mL). The mixture was then stirred at ambient temperature for 64 h. The reaction mixture was evaporated to dryness (60° C., 12 mbar) and the resulting residue triturated with DCM followed by 10% MeOH/DCM, collecting the solvent fraction by filtration. The filtrate was evaporated and the residual material subjected to chromatography over a Strata® SI-1 silica cartridge (10 g Giga™ Tube), eluting with 33% v/v EtOAc/light petroleum followed by 10% v/v MeOH/DCM. Fractions containing the product were combined and evaporated to afford 2-(2-chloro-1H-benzo[d]imidazol-1-yl)-N-(2-methoxyethyl)-acetamide (2.22 g, 8.05 mmol; 80%) as a colourless powder: δ_{H} (300 MHz, CDCl₃) 7.74-7.78 (1H, m), 7.33-7.36 (3H, m), 5.86 (1H, br s), 4.88 (2H, s), 3.45-3.50 (2H, m), 3.38-3.42 (2H, m), 3.25 (3H, s).

Step 2. A flame-dried, heavy-walled, sealable tube fitted with a magnetic stir bar was charged under argon with 2-(2-chloro-1H-benzo[d]imidazol-1-yl)-N-(2-methoxyethyl)-acetamide (134 mg, 501 μmol), pyridin-2-ylmethanamine (216 mg, 2.00 mmol) and anhydrous THF (2.5 mL). The tube was sealed and the mixture heated at 120° C. with stirring. After 18 h the mixture was cooled and evaporated to afford a residue that was subjected to chromatography over a Strata® SI-1 silica cartridge (10 g Giga™ Tube), eluting successively with 1%, 2%, 5% and 10% v/v MeOH/DCM. Fractions containing the product were combined and evaporated to afford N-(2-methoxyethyl)-2-(2-((pyridin-2-ylmethyl)amino)-1H-benzo[d]imidazol-1-yl)acetamide (SC209) (115 mg, 339 μmol; 67%) as a yellow powder: δ_{H} (300 MHz, CDCl₃) 8.55 (1H, ddd, J 4.9, 1.6 and 0.9), 7.67 (1H, td, J 7.7 and 1.8), 7.49 (1H, dt, J 7.6 and 0.9), 7.32 (1H, ˜d, J 7.8), 7.18-7.22 (1H, m), 7.09-7.17 (1H, m), 7.04-7.08 (2H, m), 6.43 (1H, br t, J 4.9), 6.20 (1H, br s), 4.80 (2H, br s), 4.64 (2H, s), 3.40-3.48 (2H, m), 3.32-3.38 (2H, m), 3.15 (3H, s); δ_{C} (300 MHz, CDCl₃) 167.1 (C), 156.3 (C), 154.4 (C), 149.0 (CH), 142.6 (C), 136.9 (CH), 134.5 (C), 122.6 (CH), 122.3 (CH), 122.1 (CH), 120.4 (CH), 116.9 (CH), 107.4 (CH), 70.8 (CH₂), 58.8 (CH₃), 47.7 (CH₂), 46.4 (CH₂), 39.5 (CH₂).

### Example 9

### N-(2-Methoxyethyl)-2-(2-(phenylamino)-1H-benzo[d]imidazol-1-yl)acetamide (SC865) was prepared according to Route 6

A flame-dried, heavy-walled, sealable tube fitted with a magnetic stir bar was charged under argon with 2-(2-chloro-1H-benzo[d]imidazol-1-yl)-N-(2-methoxyethyl)-acetamide (preparation vide supra, EXAMPLE 8; 134 mg, 501 μmol), aniline (186 mg, 2.00 mmol), KH₂PO₄ (68 mg, 0.52 mmol) and n-butanol (5 mL). The tube was sealed and the mixture heated at 80° C. with stirring. After 60 h the mixture was cooled and evaporated to afford a residue that was subjected to chromatography over a Strata® SI-1 silica cartridge (5 g Giga™ Tube), eluting successively with 33%, 50% and 75% v/v EtOAc/light petroleum followed by 5% v/v MeOH/DCM. Fractions containing the product were combined and evaporated to afford N-(2-methoxyethyl)-2-(2-(phenylamino)-1H-benzo[d]imidazol-1-yl)acetamide (SC865) (133 mg, 409 μmol; 82%) as a colourless powder: δ_{H} (300 MHz, CDCl₃) 8.27 (1H, br s), 7.48 (2H, d, J 7.2), 7.33 (2H, d, J 7.7), 7.18-7.26 (2H), 7.12 (2H, t, J 7.5), 6.92 (1H, t, J 7.1), 5.05 (2H, s), 3.41-3.50 (4H, m), 3.31 (3H, s).

### Example 10

### 2-(2-(Benzylamino)-1H-benzo[d]imidazol-1-yl)-N-(pyridin-3-yl)acetamide (SC632) was prepared according to Route 7

Step 1. To a stirred, ice-cooled solution of para-nitrophenol (1.39 g, 10.0 mmol) in anhydrous MeCN (20 mL) under argon was added triethylamine (1.46 mL, 10.5 mmol) followed dropwise by bromoacetyl bromide (915 μL, 10.5 mmol). After 20 min the mixture was removed from the ice bath and stirred at ambient temperature for 1 h. Solvent was then evaporated and the residue directly subjected to chromatography over a Strata® SI-1 silica cartridge (20 g Giga™ Tube), eluting with DCM. Fractions containing the product were combined and evaporated to afford 4-nitrophenyl 2-bromoacetate (2.56 g, 9.85 mmol; 99%) as a colourless powder: δ_{H} (300 MHz, CDCl₃) 8.28-8.33 (2H, AA′XX′), 7.32-7.37 (2H, AA′XX′), 4.08 (2H, s).

Step 2. To a stirred solution of 2-chloro-1H-benzo[d]imidazole (153 mg, 1.00 mmol) in anhydrous DMF (5 mL) at ambient temperature under argon was added NaH (60% w/w dispersion in mineral oil; 44 mg, 1.10 mmol). After 1.5 h the mixture was cooled to −40° C. prior to addition of a solution of 4-nitrophenyl 2-bromoacetate (286 mg, 1.10 mmol) in DMF (3 mL). The mixture was allowed to come to ambient temperature and stirred for 16 h. It was then diluted with DCM (25 mL) and washed successively with ice-water (2×100 mL) and brine (2×20 mL). The organic layer was dried (Na₂SO₄) and evaporated to afford a light brown solid (442 mg) that was subsequently triturated with diethyl ether, collecting the solid by filtration and discarding the ethereal phase. The solid was dried in vacuo to afford 4-nitrophenyl 2-(2-chloro-1H-benzo[d]imidazol-1-yl)acetate (221 mg, 666 μmol; 67%) as a buff powder: δ_{H} (300 MHz, CDCl₃) 8.25-8.30 (2H, AA′XX′), 7.73-7.79 (1H, m), 7.28-7.40 (5H, m), 5.21 (2H, s).

Step 3. To a solution of 3-aminopyridine (56 mg, 0.59 mmol) in DMF (3 mL) at ambient temperature under argon was added NaH (60% w/w dispersion in mineral oil; 24 mg, 0.60 mmol). After 1 h the mixture was cooled to 0° C. prior to dropwise addition of a solution of 4-nitrophenyl 2-(2-chloro-1H-benzo[d]imidazol-1-yl)acetate (166 mg, 500 μmol) in DMF (3 mL). The mixture was allowed to come to ambient temperature and stirred for 64 h. It was then evaporated to dryness (60° C., 12 mbar) and the resulting residue subjected to chromatography over a Strata® SI-1 silica cartridge (10 g Giga™ Tube), eluting successively with 1%, 2%, 5% and 10% v/v MeOH/DCM. Fractions containing the product were combined and evaporated. The resulting yellow waxy solid (58 mg) was found to contain target material and para-nitrophenol (ca. 1:1) ratio by ¹H NMR analysis. The chromatography was therefore repeated over a Strata® SI-1 silica cartridge (5 g Giga™ Tube), eluting successively with 50% v/v EtOAc/light petroleum, EtOAc and 1% v/v MeOH/DCM. Fractions containing the product were again combined and evaporated to afford 2-(2-chloro-1H-benzo[d]imidazol-1-yl)-N-(pyridin-3-yl)acetamide (31 mg; ca. 85-90% mass purity).

Step 4. The 2-(2-chloro-1H-benzo[d]imidazol-1-yl)-N-(pyridin-3-yl)acetamide (31 mg) prepared in the preceding step was taken up in anhydrous THF (2.5 mL) and transferred under argon into a flame-dried, heavy-walled, sealable tube fitted with a magnetic stir bar. Benzylamine (97 μL, 0.89 mmol) was added. The tube was sealed and the mixture heated at 120° C. with stirring. After 24 h the mixture was cooled and evaporated to afford a residue that was subjected to chromatography over a Strata® SI-1 silica cartridge (5 g Giga™ Tube), eluting successively with 0.5%, 2% and 5% v/v MeOH/DCM. Fractions containing the product were combined and evaporated to afford a residue (34 mg). The residue was triturated with CHCl₃/Et₂O, collecting the solid, to afford 2-(2-(benzylamino)-1H-benzo[c]imidazol-1-yl)-N-(pyridin-3-yl)acetamide (SC632) (17 mg) as a colourless powder: δ_{H} (300 MHz, CD₃CN) 8.86 (1H, br s), 8.81 (1H, d, J 2.5), 8.35-8.39 (1H, m), 8.08 (1H, dd, J 4.7 and 1.4), 8.05 (1H, br s), 7.31-7.34 (1H, m), 7.10-7.23 (6H, m), 6.94-7.04 (2H, m), 4.88 (2H, s), 4.35 (2H, d, J 6.0). The following compounds were prepared according to the methods outlined above.

### By Route 1:

2-(2-(benzylamino)-1H-benzo[d]imidazol-1-yl)-N-(pyridin-2-yl)acetamide (SC938)
2-(2-(benzylamino)-1H-benzo[d]imidazol-1-yl)-N-(pyridin-4-yl)acetamide (SC882)
2-(2-(benzylamino)-1H-benzo[d]imidazol-1-yl)-N-(o-tolyl)acetamide (SC498)
2-(2-(benzylamino)-1H-benzo[d]imidazol-1-yl)-N-(m-tolyl)acetamide (SC386)
2-(2-(benzylamino)-1H-benzo[d]imidazol-1-yl)-N-(p-tolyl)acetamide (SC274)
2-(2-(benzylamino)-1H-benzo[d]imidazol-1-yl)-N-(5-methylisoxazol-3-yl)acetamide (SC332)
2-(2-(benzylamino)-1H-benzo[d]imidazol-1-yl)-N-(3-methylisoxazol-5-yl)acetamide (SC330)
2-(2-(benzylamino)-1H-benzo[d]imidazol-1-yl)-N-(2-methoxyphenyl)acetamide (SC162)
2-(2-(benzylamino)-1H-benzo[d]imidazol-1-yl)-N-(3-methoxyphenyl)acetamide (SC106)
2-(2-(benzylamino)-1H-benzo[d]imidazol-1-yl)-N-(4-methoxyphenyl)acetamide (SC218)
2-(2-(benzylamino)-1H-benzo[d]imidazol-1-yl)-N-(2-methoxyethyl)acetamide (SC050)
2-(2-(benzylamino)-1H-benzo[d]imidazol-1-yl)-N-(3-methoxypropyl)acetamide (SC399)
2-(2-(benzylamino)-1H-benzo[d]imidazol-1-yl)-N-(pyrazin-2-yl)acetamide (SC119)
2-(2-(benzylamino)-1H-benzo[d]imidazol-1-yl)-N-(tetrahydrofuran-3-yl)acetamide (SC007)
2-(2-(benzylamino)-1H-benzo[d]imidazol-1-yl)-N-(2-isopropoxyethyl)acetamide (SC951)
2-(2-(benzylamino)-1H-benzo[d]imidazol-1-yl)-N-(2-ethoxyethyl)acetamide (SC839)
(S)-2-(2-(benzylamino)-1H-benzo[d]imidazol-1-yl)-N-(1-methoxypropan-2-yl)acetamide (SC727)
(R)-2-(2-(benzylamino)-1H-benzo[d]imidazol-1-yl)-N-(1-methoxypropan-2-yl)acetamide (SC321)
2-(2-(benzylamino)-1H-benzo[d]imidazol-1-yl)-N-((tetrahydrofuran-2-yl)methyl)acetamide (SC943)
2-(2-(benzylamino)-1H-benzo[d]imidazol-1-yl)-N-(2-(2-methoxyethoxy)ethyl)acetamide (SC831)
2-(2-(benzylamino)-1H-benzo[d]imidazol-1-yl)-N-(2-hydroxyethyl)acetamide (SC705)
N-benzyl-2-(2-(benzylamino)-1H-benzo[d]imidazol-1-yl)acetamide (SC649)
2-(2-(benzylamino)-1H-benzo[d]imidazol-1-yl)-N-cyclopentylacetamide (SC265)
2-(2-(benzylamino)-1H-benzo[d]imidazol-1-yl)-N-butylacetamide (SC977)
2-(2-(benzylamino)-1H-benzo[d]imidazol-1-yl)-N-(2-propoxyethyl)acetamide (SC921)
2-(2-(benzylamino)-1H-benzo[d]imidazol-1-yl)-N-(pyridin-2-ylmethyl)acetamide (SC153)
2-(2-(benzylamino)-1H-benzo[d]imidazol-1-yl)-N-(furan-2-ylmethyl)acetamide (SC537)
2-(2-(benzylamino)-1H-benzo[d]imidazol-1-yl)-N-(2-methoxybenzyl)acetamide (SC607)
2-(2-(benzylamino)-1H-benzo[d]imidazol-1-yl)-N-(isoxazol-4-yl)acetamide (SC985)
2-(2-(benzylamino)-1H-benzo[d]imidazol-1-yl)-N-(3,4-dimethylisoxazol-5-yl)acetamide (SC495)
2-(2-(benzylamino)-1H-benzo[d]imidazol-1-yl)-N-(2-methoxyethyl)-N-methylacetamide (SC929)
2-(2-(benzylamino)-1H-benzo[d]imidazol-1-yl)-N-(1,3-dimethyl-1H-pyrazol-5-yl)acetamide (SC105)
2-(2-(benzylamino)-1H-benzo[d]imidazol-1-yl)-N-(2-(trifluoromethoxy)ethyl)acetamide (SC313)
2-(2-(benzylamino)-1H-benzo[d]imidazol-1-yl)-1-morpholinoethan-1-one (SC489)
N-(2-methoxyethyl)-2-(2-methyl-1H-benzo[d]imidazol-1-yl)acetamide (SC666)
2-(2-(benzylthio)-1H-benzo[d]imidazol-1-yl)-N-(2-methoxyethyl)acetamide (SC697)
N-(2-methoxyethyl)-2-(2-phenethyl-1H-benzo[d]imidazol-1-yl)acetamide (SC352)
2-(2-(benzylamino)-1H-benzo[d]imidazol-1-yl)-N-(2-(methylamino)-2-oxoethyl)acetamide (SC257)
methyl (2-(2-(benzylamino)-1H-benzo[d]imidazol-1-yl)acetyl)glycinate (SC610)
2-(2-(benzylamino)-1H-benzo[d]imidazol-1-yl)-N,N-bis(2-methoxyethyl)acetamide 2-(2-(benzylamino)-1H-benzo[d]imidazol-1-yl)-N,N-bis(2-methoxyethyl)acetamide (SC817)2-(2-(benzylamino)-1H-benzo[d]imidazol-1-yl)-N-(cyanomethyl)acetamide (SC933) 2-(2-(benzylamino)-1H-benzo[d]imidazol-1-yl)-N-(2-phenoxyethyl)acetamide (SC201)
2-(2-(benzylamino)-1H-benzo[d]imidazol-1-yl)-N-(2-methylthiazol-5-yl)acetamide (SC377)
2-(2-(benzylamino)-1H-benzo[d]imidazol-1-yl)-N-(5-methyl-1,3,4-oxadiazol-2-yl)acetamide (SC408)
2-(2-(benzylamino)-1H-benzo[d]imidazol-1-yl)-N-(1,3,4-thiadiazol-2-yl)acetamide (SC033)
2-(2-(benzylamino)-1H-benzo[d]imidazol-1-yl)-N-(5-methyl-1,2,4-oxadiazol-3-yl)acetamide (SC554)
2-(2-(benzylamino)-1H-benzo[d]imidazol-1-yl)-N-(3-methyl-1,2,4-oxadiazol-5-yl)acetamide (SC585)
2-(2-(benzylamino)-1H-benzo[d]imidazol-1-yl)-N-(2-methyl-2H-tetrazol-5-yl)acetamide (SC761)
2-(2-(benzylamino)-1H-benzo[d]imidazol-1-yl)-N-(1-methyl-1H-tetrazol-5-yl)acetamide (SC271)

### By Route 2:

tert-butyl 2-(2-(benzylamino)-1H-benzo[d]imidazol-1-yl)acetate (SC343)
2-(2-(benzylamino)-1H-benzo[d]imidazol-1-yl)acetic acid (SC287)

### By Route 3:

N-benzyl-1-(2-methoxyethyl)-1H-benzo[d]imidazol-2-amine (SC231)
N-benzyl-1-(2-(benzyloxy)ethyl)-1H-benzo[d]imidazol-2-amine (SC175)
N-benzyl-1-(2-(2-methoxyethoxy)ethyl)-1H-benzo[d]imidazol-2-amine (SC873)

### By Route 4:

2-(2-(benzyl(methyl)amino)-1H-benzo[d]imidazol-1-yl)-N-(5-methylisoxazol-3-yl)acetamide (SC881)
2-(2-(benzyl(methyl)amino)-1H-benzo[d]imidazol-1-yl)-N-(2-methoxyethyl)acetamide (SC097)

### By Route 5:

2-(2-amino-1H-benzo[d]imidazol-1-yl)-N-(2-methoxyethyl)acetamide (SC593)
2-(2-(isobutylamino)-1H-benzo[d]imidazol-1-yl)-N-(2-methoxyethyl)acetamide (SC769)

### By Route 6:

2-(2-((4-chlorobenzyl)amino)-1H-benzo[d]imidazol-1-yl)-N-(2-methoxyethyl)acetamide (SC719)
N-(2-methoxyethyl)-2-(2-((pyridin-2-ylmethyl)amino)-1H-benzo[d]imidazol-1-yl)acetamide (SC209)
(R)—N-(2-methoxyethyl)-2-(2-((1-phenylethyl)amino)-1H-benzo[d]imidazol-1-yl)acetamide (SC481)
(S)—N-(2-methoxyethyl)-2-(2-((1-phenylethyl)amino)-1H-benzo[d]imidazol-1-yl)acetamide (SC041)
N-(2-methoxyethyl)-2-(2-(phenylamino)-1H-benzo[d]imidazol-1-yl)acetamide (SC865)
N-(2-methoxyethyl)-2-(2-(((tetrahydro-2H-pyran-4-yl)methyl)amino)-1H-benzo[d]imidazol-1-yl)acetamide (SC657)
N-(2-methoxyethyl)-2-(2-((pyridin-3-ylmethyl)amino)-1H-benzo[d]imidazol-1-yl)acetamide (SC520)
2-(2-((3-chlorobenzyl)amino)-1H-benzo[d]imidazol-1-yl)-N-(2-methoxyethyl)acetamide (SC383)
2-(2-((2-chlorobenzyl)amino)-1H-benzo[d]imidazol-1-yl)-N-(2-methoxyethyl)acetamide (SC529)
2-(2-(benzylamino)-1H-benzo[d]imidazol-1-yl)-N-(2-(dimethylamino)ethyl)acetamide (SC625)

### By Route 7:

2-(2-(benzylamino)-1H-benzo[d]imidazol-1-yl)-N-(pyrimidin-5-yl)acetamide (SC425)
2-(2-(benzylamino)-1H-benzo[d]imidazol-1-yl)-N-(pyridin-3-yl)acetamide (SC632)
2-(2-(benzylamino)-1H-benzo[d]imidazol-1-yl)-N-(pyridazin-3-yl)acetamide (SC809)
2-(2-(benzylamino)-1H-benzo[d]imidazol-1-yl)-N-(1-methyl-1H-pyrazol-5-yl)acetamide (SC369)
2-(2-(benzylamino)-1H-benzo[d]imidazol-1-yl)-N-(1-methyl-1H-pyrazol-4-yl)acetamide (SC545)
2-(2-(benzylamino)-1H-benzo[d]imidazol-1-yl)-N-(1-methyl-1H-pyrazol-3-yl)acetamide (SC576)
2-(2-(benzylamino)-1H-benzo[d]imidazol-1-yl)-N-(isoxazol-3-yl)acetamide (SC722)
2-(2-(benzylamino)-1H-benzo[d]imidazol-1-yl)-N-(pyridazin-4-yl)acetamide (SC753)

### By Other Routes

(2-(2-(benzylamino)-1H-benzo[d]imidazol-1-yl)acetyl)glycine (SC433)—By hydrolysis of SC610
2-methoxyethyl 2-(2-(benzylamino)-1H-benzo[d]imidazol-1-yl)acetate (SC089)—Route 1, adapted with 2-methoxyethyl 2-chloroacetate
2-(2-(benzylamino)-1H-benzo[d]imidazol-1-yl)-N-(2-(dimethylamino)ethyl)acetamide (SC145) Route 1, adapted with 2-chloro-N-(2-methoxyethyl)propanamide
N-(2-(2-(benzylamino)-1H-benzo[d]imidazol-1-yl)ethyl)-2-methoxyacetamide (SC683)—Route 1, adapted to start with alkylation using 2-(2-bromoethyl)isoindoline-1,3-dione followed by hydrazine treatment to liberate the primary amine and N-acylation with methoxyacetyl chloride.
2-(2-(benzylamino)-1H-imidazol-1-yl)-N-(2-methoxyethyl)acetamide (SC513)—Route 1, adapted to start from 2-(benzylamino)imidazole, which was prepared by reductive amination of the known precusor, 1-trityl-1H-imidazol-2-amine, with benzaldehyde and sodium triacetoxyborohydride followed by detritylation with trifluoroacetic acid.
   The structural formula of the above compounds are illustrated below.

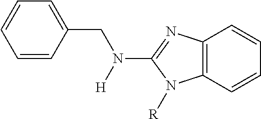

### R=

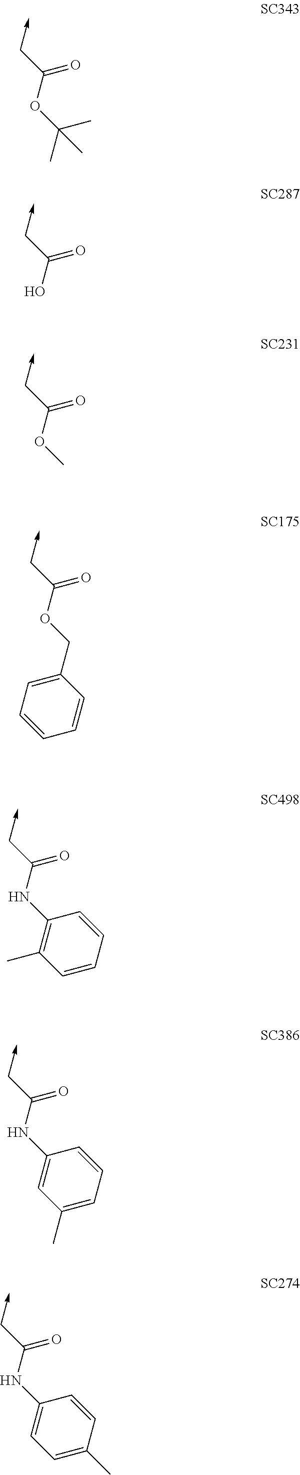

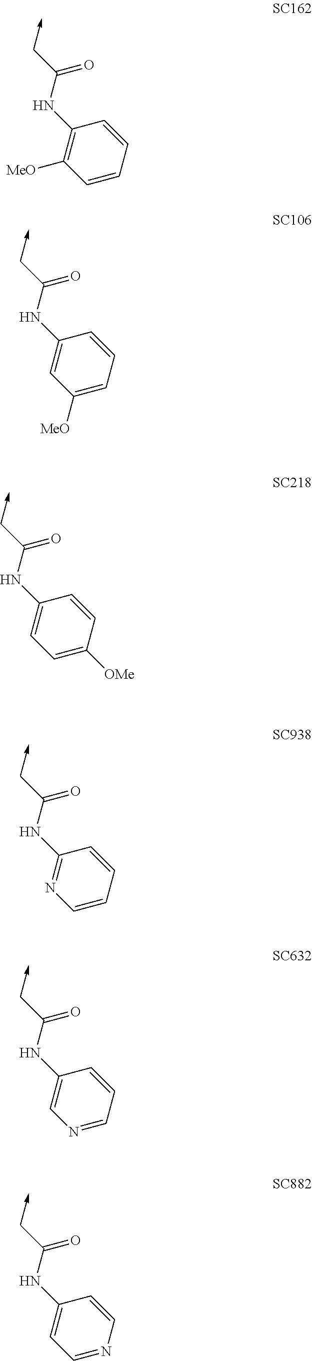

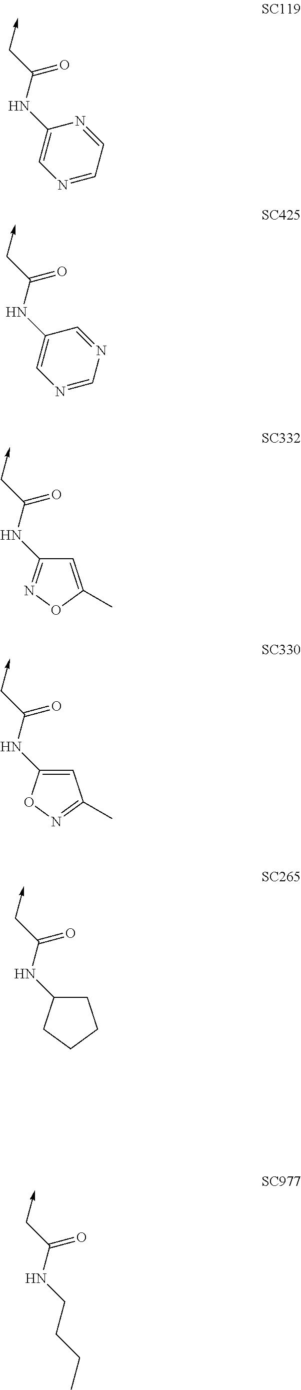

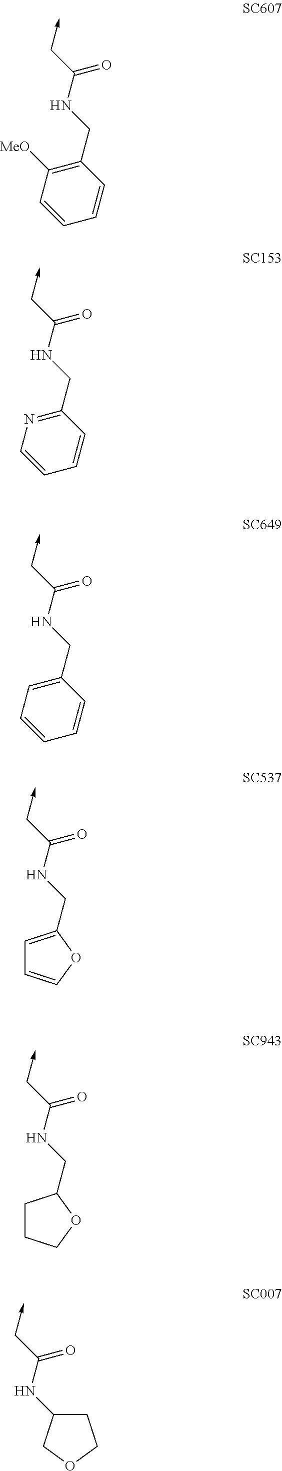

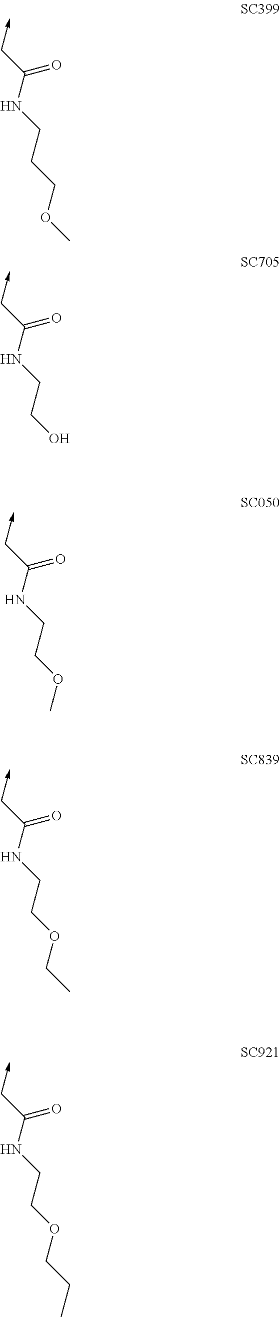

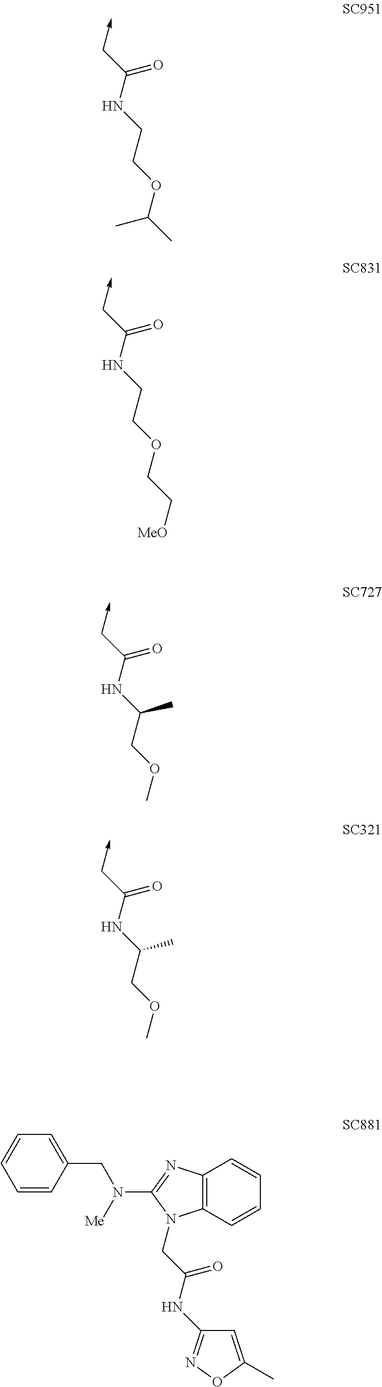

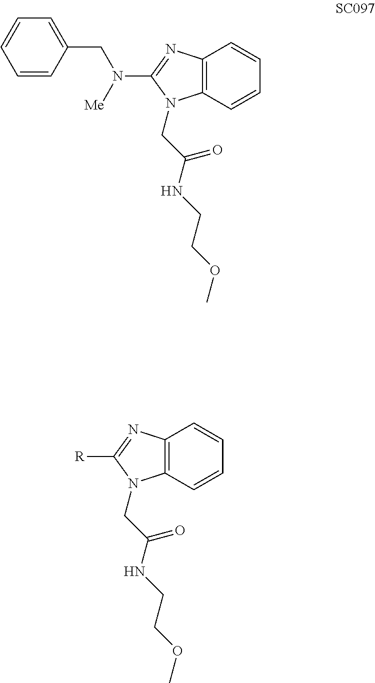

### R=

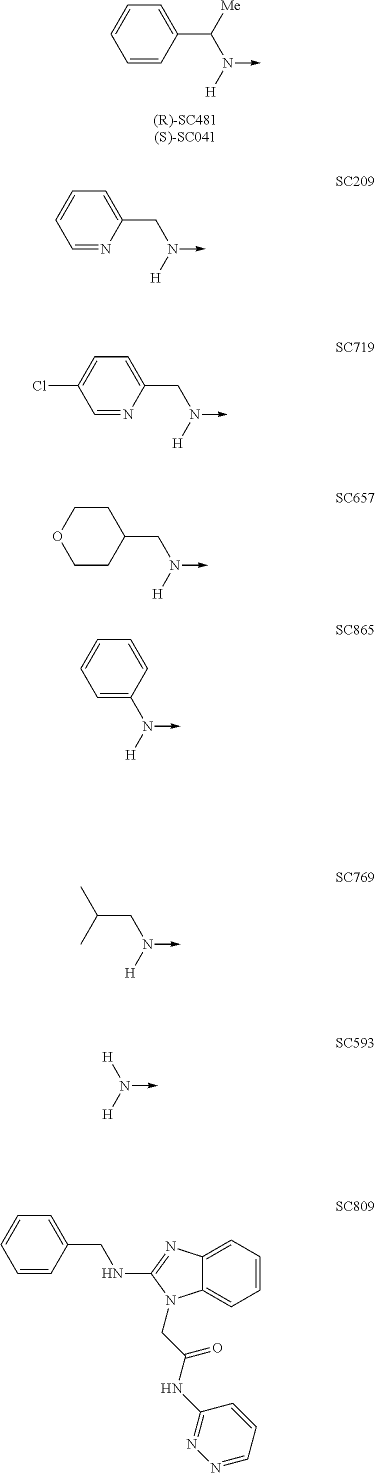

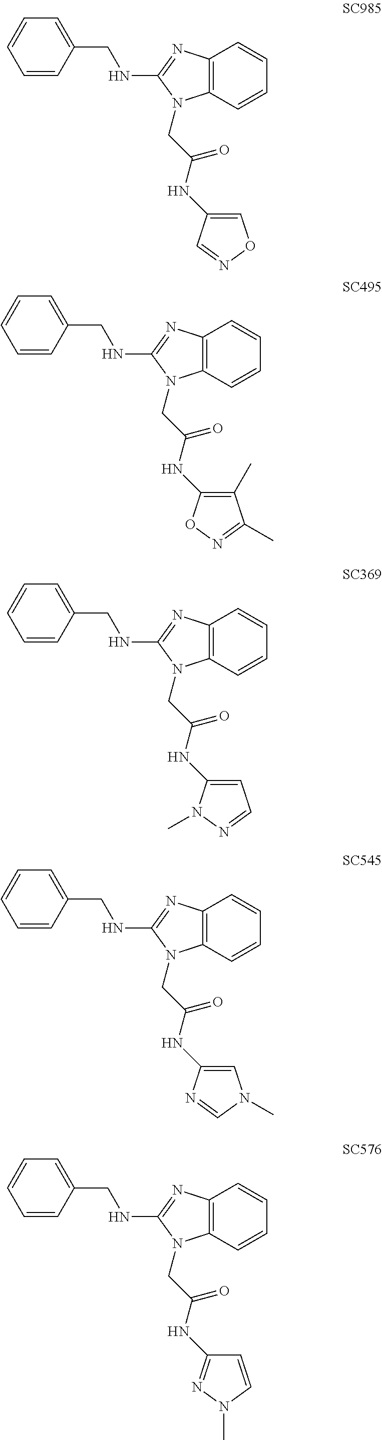 [SMILES: O=C(Cn1c(NCc2ccccc2)nc2ccccc21)Nc1cnoc1 ; Cc1noc(NC(=O)Cn2c(NCc3ccccc3)nc3ccccc32)c1C ; Cn1nccc1NC(=O)Cn1c(NCc2ccccc2)nc2ccccc21 ; Cn1cnc(NC(=O)Cn2c(NCc3ccccc3)nc3ccccc32)c1 ; Cn1ccc(NC(=O)Cn2c(NCc3ccccc3)nc3ccccc32)n1]

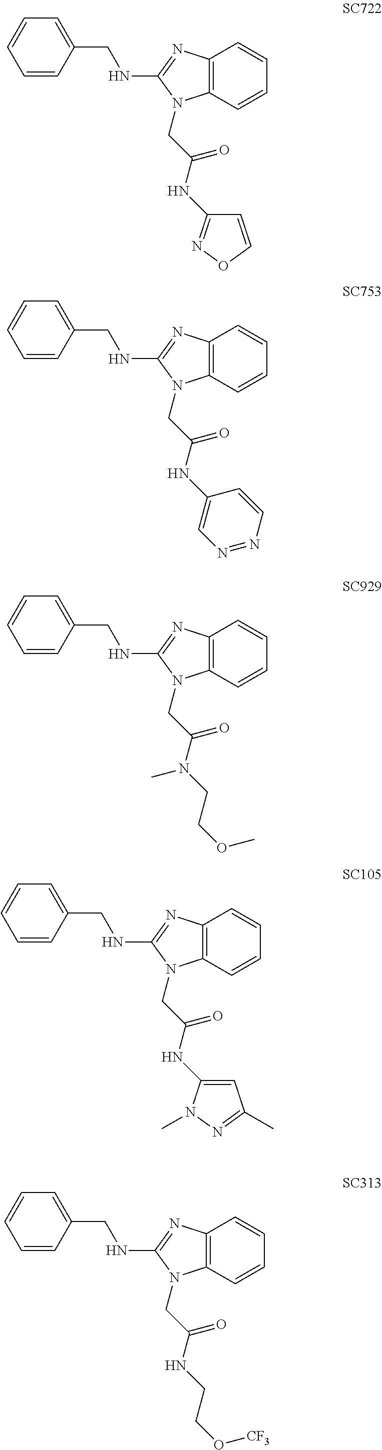 [SMILES: O=C(Cn1c(NCc2ccccc2)nc2ccccc21)Nc1ccon1 ; O=C(Cn1c(NCc2ccccc2)nc2ccccc21)Nc1ccnnc1 ; COCCN(C)C(=O)Cn1c(NCc2ccccc2)nc2ccccc21 ; Cc1cc(NC(=O)Cn2c(NCc3ccccc3)nc3ccccc32)n(C)n1 ; O=C(Cn1c(NCc2ccccc2)nc2ccccc21)NCCOC(F)(F)F]

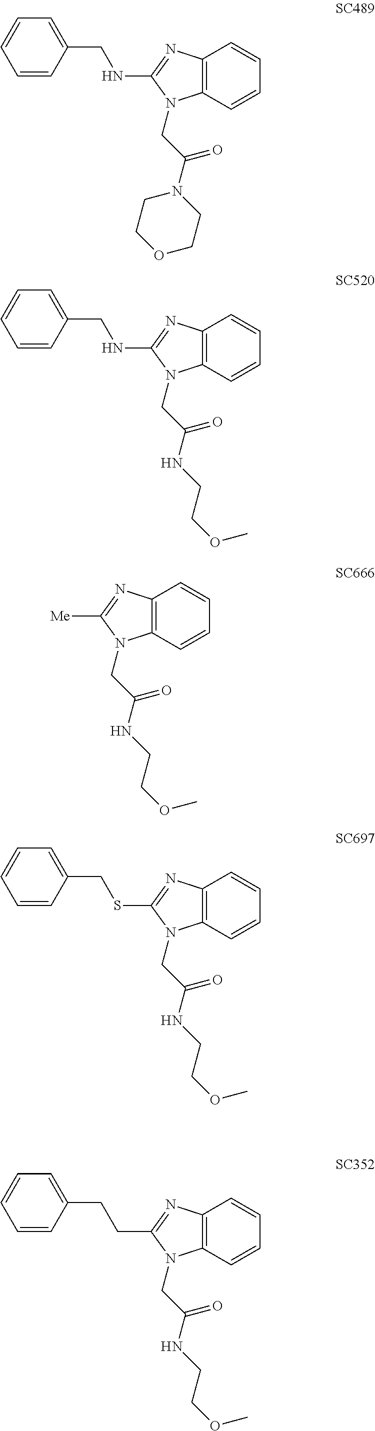 [SMILES: O=C(Cn1c(NCc2ccccc2)nc2ccccc21)N1CCOCC1 ; COCCNC(=O)Cn1c(NCc2ccccc2)nc2ccccc21 ; COCCNC(=O)Cn1c(C)nc2ccccc21 ; COCCNC(=O)Cn1c(SCc2ccccc2)nc2ccccc21 ; COCCNC(=O)Cn1c(CCc2ccccc2)nc2ccccc21]

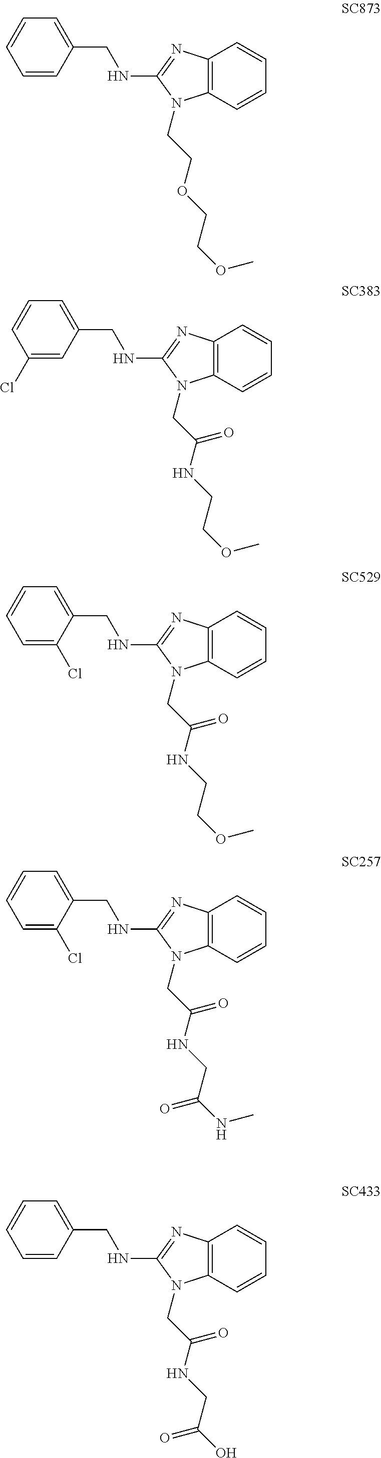 [SMILES: COCCOCCn1c(NCc2ccccc2)nc2ccccc21 ; COCCNC(=O)Cn1c(NCc2cccc(Cl)c2)nc2ccccc21 ; COCCNC(=O)Cn1c(NCc2ccccc2Cl)nc2ccccc21 ; CNC(=O)CNC(=O)Cn1c(NCc2ccccc2Cl)nc2ccccc21 ; O=C(O)CNC(=O)Cn1c(NCc2ccccc2)nc2ccccc21]

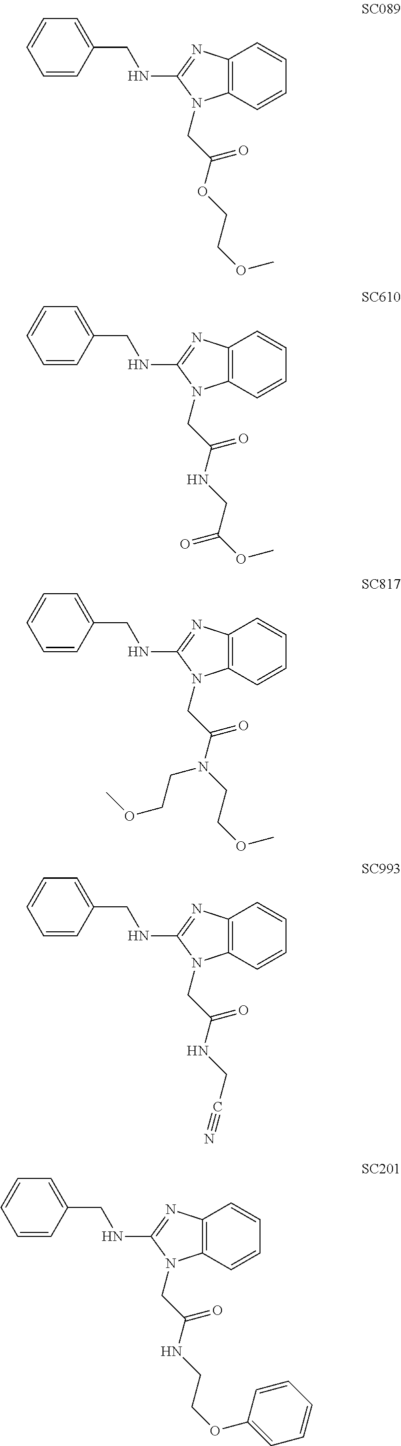 [SMILES: COCCOC(=O)Cn1c(NCc2ccccc2)nc2ccccc21 ; COC(=O)CNC(=O)Cn1c(NCc2ccccc2)nc2ccccc21 ; COCCN(CCOC)C(=O)Cn1c(NCc2ccccc2)nc2ccccc21 ; N#CCNC(=O)Cn1c(NCc2ccccc2)nc2ccccc21 ; O=C(Cn1c(NCc2ccccc2)nc2ccccc21)NCCOc1ccccc1]

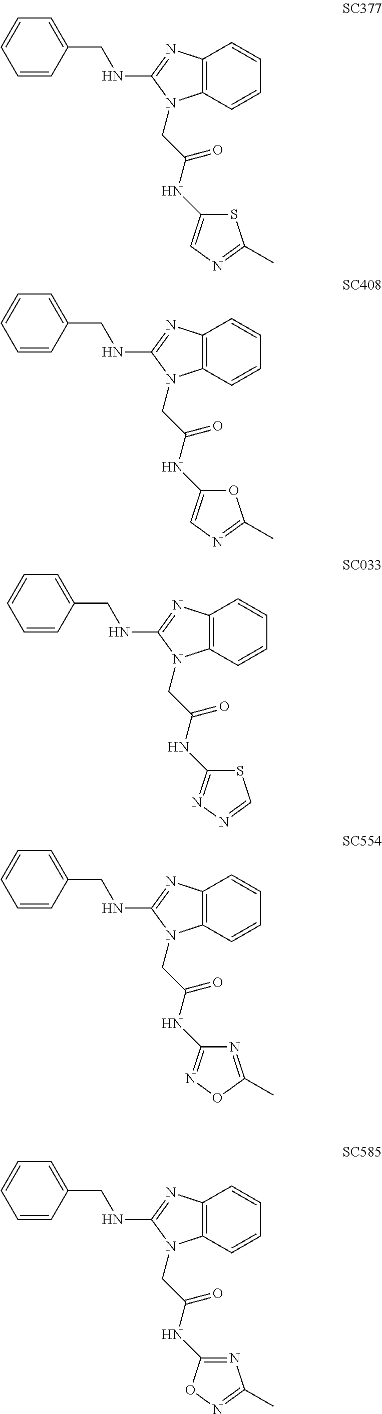 [SMILES: Cc1ncc(NC(=O)Cn2c(NCc3ccccc3)nc3ccccc32)s1 ; Cc1ncc(NC(=O)Cn2c(NCc3ccccc3)nc3ccccc32)o1 ; O=C(Cn1c(NCc2ccccc2)nc2ccccc21)Nc1nncs1 ; Cc1nc(NC(=O)Cn2c(NCc3ccccc3)nc3ccccc32)no1 ; Cc1noc(NC(=O)Cn2c(NCc3ccccc3)nc3ccccc32)n1]

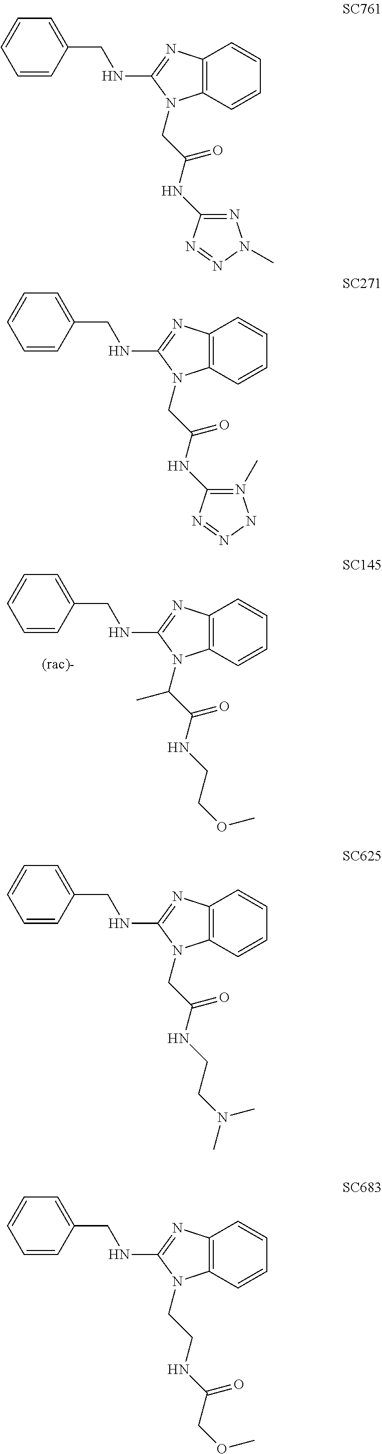

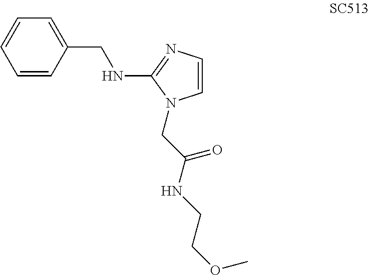 [SMILES: COCCNC(=O)Cn1ccnc1NCc1ccccc1]

### Biology

### Reagents and Materials

StemPro® hESC SFM Human Embryonic Stem Cell Culture Medium (Life Technologies)

### 50 mM 2-Mercaptoethanol (Life Technologies)

bFGF (R&D)

### PBS (−/−) (Life Technologies)

Fibronectin, Human plasma (Merck Millipore)

### TrypLE Select (1×) (Life Technologies)

H1 and H9 hESC (Thomson et al, 1998)
RC9 (Roslin cells LTD)
iPSC NMF-iPS6 (Sullivan et al, 2010)
6 well culture plates (Corning)
15 ml conical bottomed tubes (Corning)
Serological pipettes (Corning)
Stempro® EZPassage disposable passage tool (Life Technologies)
SSEA4 antibody (BD biosciences)
SSEA1 antibody (BD biosciences)
Smooth muscle actin antibody (Sigma)
Alpha fetoprotein antibody (Abcam)
PAX 6 antibody (Sigma)
α-tubulin antibody (Sigma)
pMLC antibody (Sigma)
Nitrocellulose membranes (Sigma)
4-12% Bis-Tris midi gel (Life Technologies)
MES buffer (Life Technologies)
Transfer buffer (Life Technologies)
Laemmli buffer

### Phospho-blocker (Cambridge Bioscience)

Tris buffered saline with tween (Sigma)
Enhanced Chemiluminescence (ECL) reagent (Thermo Scientific)

### Heraeus Multifuge 3 SR (Thermo Scientific)

FACS Canton II (BD biosciences)

### XCell SureLock® Mini-Cell (Life Technologies)

### Y27632 (Tocris Bioscience)

Enzymatic passage of hPSC methodology
1. 24 hrs prior to passage (around 70-80% confluence), supply 1 well of near confluent hPSC with 2 ml StemPro supplemented with 30 μM SC332 (done during usual daily feed).
2. At point of passage, remove media and wash cells 1× in PBS (−/−).
3. Add 1.5 ml of TrypLE Select (1×) to well and return to incubator for 2-5 mins to dissociate to single cells.
4. Add 1.5 ml of StemPro to deactivate TrypLE Select and pipette gently using a 5 ml serological pipette (to dissociate any remaining clumps).
5. Transfer cells to 15 ml conical bottomed tube and centrifuge for 3 mins at 300 g.
6. Aspirate as much supernatant as possible and resuspend cell pellet by gently flicking.
7. Resuspend cells in PBS (−/−) and centrifuge again for 3 mins at 300 g.
8. Aspirate PBS (−/−) and resuspend cells in 1 ml of StemPro. Gently pipette using serological pipette.
9. Perform a cell count and replate hPSC at a density of 5×10⁵ cells per well of a pre-coated 6 well plate. Top up to 2 mls total with StemPro supplemented with 30 μM SC332. Wells are pre-coated with 0.3 mg/ml fibronectin (recombinant vitronectin fragment can also be used).
10. Return cells to incubator and supply with fresh StemPro on a daily basis.
11. 24 hrs prior to subsequent passages (around 70-80% confluence), supply 1 well of PSC with 2 ml StemPro supplemented with 30 μM SC332 (done during usual daily feed).
NB SC332 can be used transiently as described above or can be added to culture medium continuously (still at 30 μM).

### Results

Human pluripotent stem cells (hPSC) were either treated with SC332, Y27632, or left untreated, before being enzymatically passaged. In all experiments untreated cells had an equivalent volume of DMSO added as a carrier control, with cell survival being assayed 24 hrs post passage. HPSC treated with either SC332 or Y27632 had average cell survival that was significantly higher than that observed in untreated controls (P=<0.001) (FIG. 1). In particular, the hESC lines H1, H9 and RC9 and the hiPSC NMF-iPSC6 had cell survival of 72% (±2.9), 73% (±2.8), 69% (±1.9) and 74% (±2.6) respectively when treated with SC332. In the same cell lines, Y27632 resulted in cell survival of 72% (±3.6), 73% (±2.1), 71% (±1.2) and 74% (±4.0) and DMSO alone resulted in survival of 18% (±2.2), 18% (±2.4), 15% (±3.0) and 19% (±2.4) respectively. This result was consistent in both hESC and iPSC. Data shown is the mean survival ±SEM, n=3.

These results confirm that compound SC332 supports the enzymatic passage of hPSC as effectively as the well-established pro-survival compound Y27632.

During long term exposure experiments, compound SC332 was shown to support enzymatic passage of hPSC as effectively as Y27632 for at least 30 consecutive passages. In addition to this, long term exposure to compound SC332 had no detrimental effect on the karyotypic stability of hPSC (FIGS. 2 and 3).

These data show that compound SC332 is a viable alternative to Y27632 as a tissue culture reagent that can be utilised over multiple passages.

It is important than any reagent used in the general maintenance of hPSC does not negatively impact upon the stem cell identity. In order to confirm this, flow cytometric analysis was performed on cells treated over multiple passages with SC332. FIG. 4 shows the % of SSEA4 (stage-specific embryonic antigen 4) and SSEA1 (stage-specific embryonic antigen 1) positive cells after each subsequent 5 passages. SSEA4 is a cell surface marker present on all undifferentiated hPSC, whereas SSEA1 is only present on differentiated cells. These markers have been routinely used as positive and negative markers of pluripotency in hPSC since their initial isolation (Thomson et al, 1998). As can be seen, consistent enzymatic passage supported by either SC332 or Y27632 has no effect on the expression level of the pluripotency marker SSEA4, with both remaining >90% positive. Furthermore the differentiation marker SSEA1 remains consistently low (expressed on <5% of cells). This shows that SC332 treated cells retain a cell surface marker profile consistent with that expected of hPSC, and comparable to that achieved with Y27632 treatment.

The differentiation capacity of hPSC after treatment with SC332 was also assessed. HPSC (hESC cell line H1 and NMF-iPS6) that had been enzymatically passaged with either SC332 or Y27632 for 30 consecutive passages were passively differentiated alongside mechanically maintained cells using a mixture of embryoid body based suspension culture and adherent culture. Differentiated cells were subsequently fixed and stained for markers from each of the three germ layers. Long term exposure to either survival compound did not block the differentiation towards mesoderm (smooth muscle actin; SMA), endoderm (alpha fetoprotein; AFP) or ectoderm (paired box protein; PAX6). As expected, untreated mechanically passaged control cells, included as a positive control, were also able to differentiate into each of the three germ layers (FIG. 5).

### ROCK Independence

To reaffirm that SC332 did not inhibit ROCK or the closely related PRK2, the compound was rescreened at the Dundee International Centre for Kinase Profiling at both the standard 10 μM and optimum 30 μM concentrations. This screen utilises a radioactive filter-binding assay (33P-ATP) to assess the effect of compounds on kinase activity. Results confirmed that SC332 had no inhibitory effect on ROCK2 at a concentration of 10 μM (96% activity. SC332 also had very limited effect on the closely related PRK2, showing 76% and 98% activity when used at 10 μM and 30 μM respectively (FIG. 6).

It has been suggested that the cause of dissociation induced apoptosis of hPSC is the hyperphosphorylation of myosin light chain (MLC) and that the protective activity of Y27632 is to prevent this phosphorylation (Chen et al, 2010; Ohgushi et al, 2010 and Walker et al, 2010), therefore western blot analysis was performed to test whether SC332 shares this biochemical effect. The data shown in FIGS. 7A and B shows that, as expected, the untreated cells had significantly higher levels of pMLC 15 mins post-dissociation and that Y27632 blocked the increase (P=<0.01), however cells treated with SC332 showed high levels of pMLC equivalent to those in untreated cells (P=>0.05). The higher levels of pMLC were maintained in untreated and SC332 treated cells compared to Y27632 treated cells at later time points, with SC332 vs Y27632 being significantly different at 30 mins (P=<0.05), 45 mins (P=<0.05) and 1 hr (P=<0.05). Although untreated cells did not reach significance versus Y27632 treated cells at these subsequent time points, the trend was towards increased levels of pMLC in untreated hPSC.

These data from untreated and Y27632 treated cells are consistent with those observed by others (Chen et al, 2010; Ohgushi et al, 2010), confirming that in response to dissociation there is a sudden increase in phosphorylated MLC and that treatment with Y27632 is able to prevent this hyperphosphorylation. Importantly though, treatment with SC332 did not inhibit the phosphorylation of MLC strongly suggesting that SC332 does not share the same mechanistic downstream target reported for Y27632. These findings also clearly uncouple the inhibition of MLC hyperphosphorylation and hPSC cell survival, supporting the hypothesis that there is an additional novel pro-survival pathway in these cells that may be elucidated using SC332.

Summary

Compounds of the invention have been found to promote survival of enzymatically dissociated hPSC. Kinase assays have confirmed that compounds of the invention do not inhibit ROCK, the reported mechanistic target of Y27632. Furthermore, biochemical analysis has shown that compounds of the invention do not produce a pro-survival effect via inhibition of MLC phosphorylation. Compounds of the invention can be used to support the passage of hPSC for at least 30 consecutive passages whilst retaining expression of pluripotency markers and normal karyotypic stability. Furthermore, cells treated with compounds of the invention retain multi-lineage differentiation capacity. Compounds of the invention are novel stem cell survival compounds with significant potential for commercial exploitation as well offering an alternative means to study the pro-survival pathways involved in dissociation induced apoptosis of hPSC.

### REFERENCES

Watanabe, K., et al., Nat Biotechnol, 2007. 25(6): p. 681-6.
Ohgushi, M., et al., Cell Stem Cell, 2010. 7(2): p. 225-39.
Ohgushi, M. and Y. Sasai, Trends Cell Biol, 2011. 21(5): p. 274-82.
Xu, Y., et al., Proc Natl Acad Sci USA, 2010. 107(18): p. 8129-34.
Andrews, P. D., et al., Biochem J, 2010. 432(1): p. 21-33.
Chen, V. C., et al., Stem Cell Res, 2012. 8(3): p. 388-402.
Amit, M., et al., Stem Cell Rev, 2010. 6(2): p. 248-59.
Singh, H., et al., Stem Cell Res, 2010. 4(3): p. 165-79.
Zweigerdt, R., et al., Nat Protoc, 2011. 6(5): p. 689-700.
Larijani, M. R., et al., Stem Cells Dev, 2011. 20(11): p. 1911-23.
Burton, P., et al., Biochem J, 2010. 432(3): p. 575-84.
Yung, S., et al., Hum Mol Genet, 2011. 20(24): p. 4932-46.
Ken K C, et al, ISSCR 2014, Abstract W2014
Krawetz, R. J., et al., PLoS One, 2011. 6(11): p. e26484.

1. A compound according to formula (I): 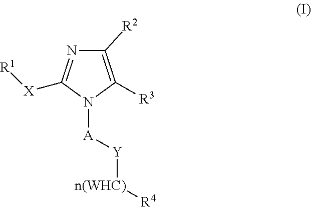
wherein:
R¹ is H, aryl, substituted aryl, alkyl, CH(CH₃)R⁵, or CH₂R⁵;
R⁵ is aryl, substituted aryl, heteroaryl, substituted heteroaryl or heterocyclic;
X is NZ, O, CH₂ or S, or alternatively X may not be present;
Z is H or alkyl;
R² and R³ are each independently selected from H, alkyl, aryl, substituted aryl, heteroaryl, substituted heteroaryl, or alternatively R² and R³ together form an aromatic or heteroaromatic ring, optionally comprising one or more substituents;
A is CH₂, CH₂CH₂ or CH(CH₃);
Y is C(═O)NZ′, C(═O)O, SO₂NH, NHC(═O), C(═O)NHC(═O), NH, or 0;
Z′ is H, alkyl, alkoxyalkyl, or wherein Z′ and R⁴ together form a 5- or 6-membered ring heterocyclic group with at least one N atom;
n is 0, 1, 2 or 3;
W is H or alkyl, wherein when n is greater than 1, each W is independently selected from H or alkyl;
R⁴ is H, alkyl, cycloalkyl, aryl, substituted aryl, heteroaryl, substituted heteroaryl, OR⁶, heterocyclic, alkylamino, C≡N or C(═O)R⁷;
R⁶ is H, alkyl, haloalkyl, aryl, alkoxyalkyl; and
R⁷ is hydroxy, alkoxy, amino or alkylamino.

2. A compound according to formula (II), (III) or (IV): 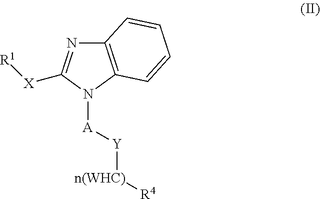
wherein R¹, X, A, Y, W, n and R⁴ are as defined for formula (I); 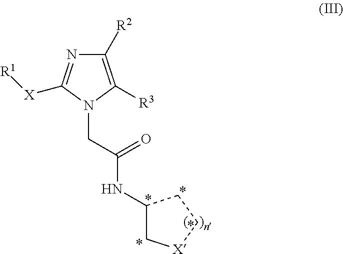
wherein R¹, R², R³ and X are as defined for formula (I);
X′ is a hydrogen bond acceptor site;
n′ is 1 or 2;
the dotted lines indicate that the hydrogen bond acceptor site is incorporated into a heteroaryl or heterocyclic ring, or alternatively the dotted lines are not present and the hydrogen bond acceptor site is present on an acyclic side chain, in either case the cyclic or acyclic side chain optionally comprises one or more alkyl substituents; and
wherein one or more of the starred carbon atoms (*) is substituted with a heteroatom; 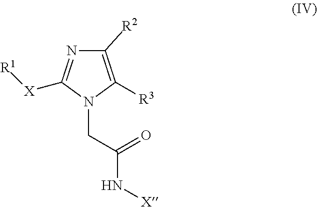
wherein R¹, R², R³ and X and are as defined for formula (I); and
X″ is a heteroaryl, substituted heteroaryl or an alkoxyalkyl group.

3. A compound having the following structure:

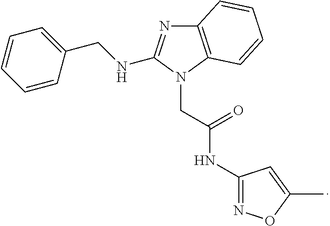 [SMILES: Cc1cc(NC(=O)Cn2c(NCc3ccccc3)nc3ccccc32)no1]

4. A composition comprising one or more compounds according to claim 1.

5.-12. (canceled)

13. A composition comprising one or more compounds according to claim 2.

14. A composition comprising one or more compounds according to claim 3.

15. A cell culture medium or cell culture media supplement comprising one or more compounds according to claim 1.

16. A cell culture medium or cell culture media supplement comprising one or more compounds according to claim 2.

17. A cell culture medium or cell culture media supplement comprising one or more compounds according to claim 3.

18. A method of maintaining a cell or cells in culture, said method comprising contacting the cell or cells with one or more compounds according to claim 1.

19. A method of maintaining a cell or cells in culture, said method comprising contacting the cell or cells with one or more compounds according to claim 2.

20. A method of maintaining a cell or cells in culture, said method comprising contacting the cell or cells with one or more compounds according to claim 3.

21. The method of claim 18, wherein the cell or cells is a stem cell or population of stem cells.

22. The method of claim 19, wherein the cell or cells is a stem cell or population of stem cells.

23. The method of claim 20, wherein the cell or cells is a stem cell or population of stem cells.

24. A kit for maintaining stem cells in culture and/or for use in a method of maintaining stem cells in culture as described herein, said kit comprising a compound according to claim 1

25. The kit of claim 24, wherein the kit further comprises one or more components selected from the group consisting of:
(a) receptacles for the culture and/or maintenance of stem cells, embryoid bodies and/or cells;
(b) tools and/or implements for adding supplements to media; and
(c) instructions for use.

26. A kit for maintaining stem cells in culture and/or for use in a method of maintaining stem cells in culture as described herein, said kit comprising a compound according to claim 2.

27. The kit of claim 26, wherein the kit further comprises one or more components selected from the group consisting of:
(a) receptacles for the culture and/or maintenance of stem cells, embryoid bodies and/or cells;
(b) tools and/or implements for adding supplements to media; and
(c) instructions for use.

28. A kit for maintaining stem cells in culture and/or for use in a method of maintaining stem cells in culture as described herein, said kit comprising a compound according to claim 3.

29. The kit of claim 26, wherein the kit further comprises one or more components selected from the group consisting of:
(a) receptacles for the culture and/or maintenance of stem cells, embryoid bodies and/or cells;
(b) tools and/or implements for adding supplements to media; and
(c) instructions for use.
